(19) United States
(12) Patent Application Publication
(10) Pub. No.: US 2026/0249546 A1
(43) Pub. Date: Aug. 27, 2026

(54) Polymeric Structures Having a Micro-Void Space and Systems and Methods for Making Same

(71) Applicant: The Board of Trustees of the Leland Stanford Junior University, Stanford, CA (US)

(72) Inventors: Joseph M. DeSimone, Stanford, CA (US); Ian A. Coates, Stanford, CA (US); Gabriel E. Lipkowitz, Redwood City, CA (US)

(21) Appl. No.: 19/120,434

(22) PCT Filed: Oct. 16, 2023
(86) PCT No.: PCT/US2023/035203

§ 371 Date: Apr. 11, 2025

Related U.S. Application Data
(60) Provisional application No. 63/416,789, filed on Oct. 17, 2022.

(51) Int. Cl. B29C 64/129; B29C 64/291; B29C 64/393; B33Y 10/00; B33Y 30/00; B33Y 50/02; B33Y 80/00

(52) U.S. Cl. CPC B29C 64/129; B29C 64/291; B29C 64/393; B33Y 10/00; B33Y 30/00; B33Y 50/02; B33Y 80/00

(57) ABSTRACT

Aspects of the present disclosure include methods for making a polymeric structure having a micro-void space. Methods according to certain embodiments include irradiating a polymerizable composition positioned between a build elevator and a build surface to generate a polymerizable composition having a polymerized region of the polymerizable composition having a micro-void space in contact with the build elevator and a non-polymerized region of the polymerizable composition in contact with the build surface, displacing the build elevator away from the build surface, contacting the generated micro-void space with a non-reactive composition and repeating in a manner sufficient to generate a polymeric structure having a resolved micro-void space. Systems for preparing a polymeric structure according to the subject methods are also described. Polymeric structures having a resolved micro-void space such as where the micro-void space is filled with a non-polymerizable composition are also provided.

### CROSS-REFERENCE TO RELATED APPLICATIONS

Pursuant to 35 U.S.C. § 119(e), this application claims priority to the filing date of U.S. Provisional Patent Application Ser. No. 63/416,789 filed Oct. 17, 2022, the disclosure of which application is incorporated herein by reference in its entirety. This application is related to U.S. Provisional Patent Application Ser. No. 63/321,052 filed Mar. 17, 2022, the disclosure of which application is herein incorporated by reference.

### GOVERNMENT RIGHTS

This invention was made with Government support under contract NSF GRFP (FELLOWSHIP) awarded by the National Science Foundation. The Government has certain rights in the invention.

### INTRODUCTION

Additive manufacturing techniques for printing polymeric resins have been used in applications such as personalized human protection, wearable electronics, functionally graded materials. Printed materials have been shown to have desirable mechanical, electrical, and chemically stable properties. Continuous liquid interface production (CLIP) like other digital light projection (DLP) methods, projects a rapid sequence of ultraviolet patterns (UV) to photopolymerize a resin layer-by-layer. Other DLP methods which require layer by layer delamination between each exposure. CLIP generates a polymer structure by resin renewal underneath the build surface through a continuous liquid interface, the dead zone, created by oxygen, a polymerization inhibitor, fed through the highly oxygen permeable window at the bottom of the resin reservoir. The combination of improved optical projection and CLIP technology has allowed printers to reach sub-micron lateral (XY) resolution at speeds 100 times faster than other 3D printing methods.

The ability to achieve a high Z resolution is needed for fabrication of negative spaces (channels, voids, etc.), an overarching characteristic of microelectronic, microsensor and microfluidic devices. Despite CLIPs ability to resolve sub-micron features in the XY plane, its ability to resolve features on this scale in the build (Z) direction is severely limited in negative feature size. This is due to UV light penetrating previously fabricated layers causing polymerization of trapped unpolymerized resin in negative spaces.

### SUMMARY

The inventors of the present disclosure have that discovered that cure-through (e.g., UV penetration) of injecting reactive polymerizable composition (e.g., through a channel to the build surface) can result in trapped polymerizable resin when forming void space in a building polymeric structure. This cure-through can hinder or prevent the formation of micro-void spaces in a polymeric structure generated by continuous liquid interface production (including injection continuous liquid interface production) The present disclosure eliminates negative polymerization and print-through effects of UV penetration.

Aspects of the present disclosure include methods for making a polymeric structure having a micro-void space. Methods according to certain embodiments include irradiating a polymerizable composition positioned between a build elevator and a build surface to generate a polymerizable composition having a polymerized region of the polymerizable composition having a micro-void space in contact with the build elevator and a non-polymerized region of the polymerizable composition in contact with the build surface, displacing the build elevator away from the build surface, contacting the generated micro-void space with a non-reactive composition and repeating in a manner sufficient to generate a polymeric structure having a resolved micro-void space. Systems for preparing a polymeric structure according to the subject methods are also described. Polymeric structures having a resolved micro-void space such as where the micro-void space is filled with a non-polymerizable composition are also provided.

In practicing the subject methods according to some embodiments, an amount of the polymerizable composition is conveyed through the generated micro-void space in a manner sufficient to displace any polymerized material (e.g., trapped resin) in the micro-void space. In some instances, the polymerizable composition is injected into the micro-void space, such as where the micro-void space is a microchannel formed within the polymeric structure. In certain instances, conveying the polymerizable composition is sufficient to flush out trapped resin in the micro-void space in order to preserve the negative space and eliminate print-through of the polymeric structure. In some instances, the polymerizable composition is continuously conveyed through the generated micro-void space into the space between the build elevator and the build surface of the liquid interface production module. In some instances, the non-reactive composition is continuously conveyed (e.g., through a conduit) into the micro-void space while displacing the build elevator away from the build surface when generating the polymeric structure.

In some embodiments, methods include contacting the generated micro-void space with a non-polymerizable composition. In some instances, the non-polymerizable composition is continuously contacted with the micro-void space while generating the polymeric structure. In some embodiments, methods include filling at least a part of the void volume of the micro-void space with the non-polymerizable composition. In some embodiments, methods include filling 5% or more of the void volume of the micro-void space with the non-polymerizable composition, such as 10% or more, such as 25% or more, such as 50% or more and including 75% or more. In some embodiments, methods include filling the entire void volume of the micro-void space with the non-polymerizable composition. In certain instances, the non-polymerizable composition is non-reactive with the polymerizable composition of the polymeric structure. In some instances, the micro-void space includes a microchannel within the polymeric structure, such as where the microchannel extends through the polymeric structure. In some instances, the polymeric structure includes a plurality of micro-void spaces. In some instances, the non-polymerizable composition is a composition selected from water, a Newtonian liquid, a shear thinning liquid, a shear thickening liquid, a magnetorheological liquid, an electric field responsive liquid and a gas.

In some embodiments, the polymerizable composition is in contact with the build elevator and the build surface. In some instances, the method includes irradiating the polymerizable composition for a duration sufficient to bond the first polymerized region of the polymerizable composition to the build elevator. In some instances, the build elevator is displaced in predetermined increments of from 0.5 μm to 1.0 μm. In some instances, the methods include adding polymerizable composition to the build surface after each displacement of the build elevator away from the build surface. In some instances, methods include continuously adding polymerizable composition to the build surface. In some instances, the polymerizable composition is continuously added to the build surface by injection through a conduit. In some embodiments, the method includes contacting the non-reactive composition (e.g., through the conduit) to the generated micro-void space after each displacement of the build elevator away from the build surface. In some embodiments, the polymerizable composition is continuously polymerized while displacing the build elevator away from the build surface. In some embodiments, the non-reactive composition is continuously conveyed through the micro-void space (e.g., microchannels formed within the polymeric structure) while displacing the build elevator away from the build surface. In some embodiments, the non-reactive composition is injected (e.g., continuously) into the micro-void space through a conduit. In some instances, methods include continuously adding non-reactive composition to the build region. In some instances, the non-reactive composition is continuously added to the build region by injection through a conduit. In certain embodiments, methods include removing the non-reactive composition (e.g., a non-polymerizable composition) from the generated micro-void space of the polymeric structure.

Aspects of the disclosure also include systems for making a polymeric structure have a micro-void space. Systems according to certain embodiments include a light source and a light interface polymerization module having a build elevator and a build surface configured for generating a polymeric structure having a resolved micro-void space therein from a polymerizable composition positioned therebetween. In some embodiments, the light interface polymerization module is configured for generating a polymeric structure having one or more microchannels in the polymeric structure. In some instances, one or more of the microchannels extends through the polymeric structure. In some embodiments, systems include a processor having memory operably coupled to the processor where the memory includes instructions stored thereon, which when executed by the processor, cause the processor to a) irradiate a polymerizable composition positioned between a build elevator and a build surface to generate a polymerizable composition having a polymerized region of the polymerizable composition with a micro-void space in contact with the build elevator and a non-polymerized region of the polymerizable composition in contact with the build surface; b) displace the build elevator away from the build surface; c) contact the generated micro-void space with a non-reactive composition; and d) repeat steps a)-c) in a manner sufficient to generate a polymeric structure having a resolved micro-void space. In some instances, systems are configured to generate a polymeric structure having a micro-void space that includes a non-polymerizable composition positioned therein.

In some embodiments, the memory includes instructions for contacting the generated micro-void space with an amount of the polymerizable composition in a manner sufficient to displace polymerized material in the micro-void space. In some instances, the memory includes instructions for continuously conveying the polymerizable composition through the generated micro-void space to displace polymerized material in the micro-void space. In some instances, the memory includes instructions for injecting polymerizable composition into the generated micro-void space to displace polymerized material in the micro-void space. In some instances, the polymerizable composition is injected into the generated micro-void space with a syringe. In certain instances, systems further include a syringe pump. In some instances, the memory includes instructions for continuously conveying the polymerizable composition through the generated micro-void space into the space between the build elevator and the build surface of the liquid interface production module.

In some instances, the system further includes a source of a non-polymerizable composition that is operably coupled to the light interface polymerization module such that the non-polymerizable composition is continuously contacted with the micro-void space while generating the polymeric structure. In some instances, the source is in communication with the build region of the light interface polymerization module through a conduit. In some instances, the non-polymerizable composition is provided to the build region through a conduit such as where the non-polymerizable composition is injected through a conduit to the build region. In some instances, the memory includes instructions for filling at least a part of the void volume of the micro-void space, such as 5% or more, such as 10% or more, such as 25% or more, such as 50% or more and including 75% or more of the void volume of the micro-void space with the non-polymerizable composition, such as for filling the entire void volume of the micro-void space with the non-polymerizable composition. In some embodiments, the memory includes instructions for generating a polymeric structure having a plurality of micro-void spaces. In certain instances, the memory includes instructions for irradiating the polymerizable composition for a duration sufficient to bond the first polymerized region of the polymerizable composition to the build elevator. In some instances, the memory includes instructions for displacing the build elevator in predetermined increments of from 0.5 μm to 1.0 μm. In some embodiments, the memory includes instructions for adding polymerizable composition to the build surface after each displacement of the build elevator away from the build surface. In some instances, the memory includes instructions for adding non-polymerizable composition to the generated micro-void space after each displacement of the build elevator away from the build surface. In some instances, the memory includes instructions for continuously polymerizing the polymerizable composition while displacing the build elevator away from the build surface. In some embodiments, the memory includes instructions for continuously adding non-polymerizable composition to the generated micro-void space while displacing the build elevator away from the build surface. In certain embodiments, the memory includes instructions for removing the non-polymerizable composition from the generated micro-void space of the polymeric structure.

In certain embodiments, the system includes a micro-digital light projection system having a light beam generator component and a light projection monitoring component. In some embodiments, the light beam generator includes two projection lenses, such as magnification lenses. In some embodiments, the light projection monitoring component includes a photodetector, such as a charge-coupled device (CCD).

Aspects of the present disclosure also include polymeric structures having a micro-void space that includes a non-polymerizable composition positioned therein. In some instances, the non-polymerizable composition fills 75% or more of the void volume of the micro-void space, such as where the non-polymerizable composition fills the entire volume of the void volume of the micro-void space. In some instances, the non-polymerizable composition is non-reactive with the polymeric structure. In some instances, the micro-void space is a microchannel within the polymeric structure, such as one that extends through the polymeric structure. In some instances, the polymeric structure includes a plurality of micro-void spaces. In some instances, the non-polymerizable composition is selected from water, a Newtonian liquid, a shear thinning liquid, a shear thickening liquid, a magnetorheological liquid, an electric field responsive liquid and a gas. In some instances, the polymeric structure is formed from a polymerizable material such as polycaprolactone, polyglycolic acid, polylactic acid, polylactic-co-glycolic acid, polyethylene glycol, polyethylene glycol dimethacrylate (PEGDMA), thiol-enes, anhydrides, polyacrylic acid, poly methylmethacrylate, trimethylolpropane triacrylate (TMPTA) monomer, polyvinyl alcohol, polyvinylpyrrolidone, vinyl carbonates, vinyl esters, acrylamides, hyaluronic acid, chitosan, collagen, gelatin, carboxymethylcellulose, and blends or copolymers thereof. In certain embodiments, aspects of the present disclosure include a polymeric structure having a resolved micro-void space where the non-polymerizable composition has been removed.

Aspects of the present disclosure also include non-transitory computer readable storage medium for making a polymeric structure in a liquid interface production module. In some instances, the non-transitory computer readable storage medium has instructions stored thereon that include: algorithm for irradiating a polymerizable composition positioned between a build elevator and a build surface to generate a polymerizable composition having a polymerized region of the polymerizable composition with a micro-void space in contact with the build elevator and a non-polymerized region of the polymerizable composition in contact with the build surface, algorithm for displacing the build elevator away from the build surface, algorithm for contacting the generated micro-void space with a non-reactive composition and algorithm for repeating one or more steps in a manner sufficient to generate a polymeric structure having a resolved micro-void space. In some instances, the non-transitory computer readable storage medium has algorithm for injecting the polymerizable composition through the conduit with a syringe pump. In some instances, the non-transitory computer readable storage medium has algorithm for generating a polymeric structure having a micro-void space that includes a non-polymerizable composition positioned therein.

In some embodiments, the non-transitory computer readable storage medium has algorithm for contacting the generated micro-void space with an amount of the polymerizable composition in a manner sufficient to displace polymerized material in the micro-void space. In some instances, the non-transitory computer readable storage medium has algorithm for continuously conveying the polymerizable composition through the generated micro-void space to displace polymerized material in the micro-void space. In some instances, the non-transitory computer readable storage medium has algorithm for injecting polymerizable composition into the generated micro-void space to displace polymerized material in the micro-void space. In some instances, the non-transitory computer readable storage medium has algorithm for injecting the polymerizable composition into the generated micro-void space with a syringe. In some instances, the non-transitory computer readable storage medium has algorithm for continuously conveying the polymerizable composition through the generated micro-void space into the space between the build elevator and the build surface of the liquid interface production module.

In some instances, the non-transitory computer readable storage medium has algorithm for contacting a non-polymerizable composition with the generated micro-void space of the polymeric structure. In some instances, the non-transitory computer readable storage medium has algorithm for filling at least a part of the void volume of the micro-void space, such as 5% or more, such as 10% or more, such as 25% or more, such as 50% or more and including 75% or more of the void volume of the micro-void space with the non-polymerizable composition, such as for filling the entire void volume of the micro-void space with the non-polymerizable composition. In some embodiments, the non-transitory computer readable storage medium has algorithm for generating a polymeric structure having a plurality of micro-void spaces. In certain instances, the non-transitory computer readable storage medium has algorithm for irradiating the polymerizable composition for a duration sufficient to bond the first polymerized region of the polymerizable composition to the build elevator. In some instances, the non-transitory computer readable storage medium has algorithm for displacing the build elevator in predetermined increments of from 0.5 μm to 1.0 μm. In some embodiments, the non-transitory computer readable storage medium has algorithm for adding polymerizable composition to the build surface after each displacement of the build elevator away from the build surface. In some instances, the non-transitory computer readable storage medium has algorithm for adding non-polymerizable composition to the generated micro-void space after each displacement of the build elevator away from the build surface. In some instances, the non-transitory computer readable storage medium has algorithm for continuously polymerizing the polymerizable composition while displacing the build elevator away from the build surface. In some embodiments, the non-transitory computer readable storage medium has algorithm for continuously adding non-polymerizable composition to the generated micro-void space while displacing the build elevator away from the build surface. In certain embodiments, the non-transitory computer readable storage medium has algorithm for removing the non-polymerizable composition from the generated micro-void space of the polymeric structure.

### BRIEF DESCRIPTION OF THE FIGURES

The invention may be best understood from the following detailed description when read in conjunction with the accompanying drawings. Included in the drawings are the following figures:

FIG. 1 depicts injecting a non-polymerizable composition into a generated micro-void space (e.g., a microchannel) according to certain embodiments.

FIG. 2A depicts a schematic of conventional CLIP 3D printing approaches and the resulting print-through effect.

FIG. 2B depicts a process for generating a polymeric structure according to certain embodiments of the present disclosure and the resulting resolved micro-void spaces (i.e., negative structures).

FIG. 2C depicts the relationship between light penetration depth and minimum channel height of a process for generating a polymeric structure according to certain embodiments and other digital light processing systems.

FIG. 2D depicts generated polymeric structures according to certain embodiments including microfluidic distributer, vascular perfusion beds, and a microfluidic-enabled microarray patch, with channels filled with dye for contrast. Scale bar 5 mm.

FIGS. 3A-3D depict a comparison between model and experimental print-through effects according to certain embodiments. FIG. 3A depicts the UV light accumulation in the formed microchannel (i.e., micro-void space) which results in a print-through effect. FIG. 3B depicts the resulting polymeric structure of FIG. 3A where the micro-void space is filled. FIG. 3C depicts UV light accumulation when polymerizable composition is continuously conveyed through the forming microchannel of the polymeric structure. FIG. 3D depicts the resulting polymeric structure of FIG. 3C where the sinuous micro-void space is resolved.

FIGS. 4A-4D depict the mitigation of print-through when generating polymeric structures having varying microfluidic channel geometries and sizes according to certain embodiments. FIG. 4A depicts different microchannel pitch in polymeric structures generated with (iCLIP) and without (CLIP) injecting polymerizable composition through the microchannel during fabrication. FIG. 4B depicts different microchannel diameters in polymeric structures generated with (iCLIP) and without (CLIP) injecting polymerizable composition through the microchannel during fabrication. FIG. 4C depicts the resolution of varying microchannel pitch geometries generated with (iCLIP) and without (CLIP) injecting polymerizable composition through the microchannel during fabrication. FIG. 4D depicts the resolution of varying microchannel diameters in polymeric structures generated with (iCLIP) and without (CLIP) injecting polymerizable composition through the microchannel during fabrication. All scale bars are 1 mm.

FIGS. 5A-5D depict microchannel resolution in relation to resin turnover when generating polymeric structures having microchannels according to certain embodiments. FIG. 5A depicts the resolution of a resin with penetration depth of 237 μm as a function of turnover number. FIG. 5B depicts the resolution of varying microchannel sizes as a function of turnover number. FIG. 5C depicts the resolution of varying microchannel geometries as a function of turnover number. FIG. 5D depicts the relationship between resin penetration depth and minimum turnover rate.

FIGS. 6A-6F depict polymeric structures having micro-void spaces (e.g., microchannels) positioned therein generated according to certain embodiments. FIG. 6A depicts a microfluidic microneedle patch. FIG. 6B depicts a microneedle patch with interconnected microfluidic channels. FIG. 6C depicts a microfluidic inductor back-filled with conductive gallium. FIG. 6D depicts a microfluidic microneedle patch with 3D micromixer. FIG. 6E depicts a vascular perfusion chamber. FIG. 6F depicts porous media separation columns with varying void fraction unit cells. All scale bars are 1 mm.

### DETAILED DESCRIPTION

Aspects of the present disclosure include methods for making a polymeric structure having a micro-void space. Methods according to certain embodiments include irradiating a polymerizable composition positioned between a build elevator and a build surface to generate a polymerizable composition having a polymerized region of the polymerizable composition having a micro-void space in contact with the build elevator and a non-polymerized region of the polymerizable composition in contact with the build surface, displacing the build elevator away from the build surface, contacting the generated micro-void space with a non-reactive composition and repeating in a manner sufficient to generate a polymeric structure having a resolved micro-void space. Systems for preparing a polymeric structure according to the subject methods are also described. Polymeric structures having a resolved micro-void space such as where the micro-void space is filled with a non-polymerizable composition are also provided.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

Methods for Making a Polymeric Structure with a Micro-Void Space

Aspects of the disclosure also include methods for making a polymeric structure having a micro-void space. Methods according to certain embodiments include irradiating a polymerizable composition positioned between a build elevator and a build surface to generate a polymerizable composition having a polymerized region of the polymerizable composition having a micro-void space in contact with the build elevator and a non-polymerized region of the polymerizable composition in contact with the build surface, displacing the build elevator away from the build surface, contacting the generated micro-void space with a non-reactive composition and repeating in a manner sufficient to generate a polymeric structure having a resolved micro-void space. These steps are repeated in a manner sufficient to generate a polymeric structure having a resolved micro-void space. For example, the steps may be repeated 2 or more times, such as 3 or more times, such as 4 or more times, such as 5 or more times, such as 10 or more times, such as 20 or more times, such as 30 or more times, such as 40 or more times, such as 50 or more times, such as 100 or more times, such as 250 or more times, such as 500 or more times and including 1000 or more times. In certain instances, the generated polymeric structure has a non-polymerizable composition positioned therein.

In some embodiments, the polymerizable composition is irradiated with a light beam generator component of a micro-digital light projection system. In some instances, the light source is a broadband light source that emits light having wavelengths from 400 nm to 1000 nm. In some instances, the broadband light source is a halogen lamp, deuterium arc lamp, xenon arc lamp, stabilized fiber-coupled broadband light source, a broadband LED with continuous spectrum, superluminescent emitting diode, semiconductor light emitting diode, wide spectrum LED white light source, a multi-LED integrated white light source, among other broadband light sources or any combination thereof. In some instances, the light source is a narrow band light source emitting a particular wavelength or a narrow range of wavelengths. In some instances, the narrow band light sources emit light having a narrow range of wavelengths, such as for example, 50 nm or less, such as 40 nm or less, such as 30 nm or less, such as 25 nm or less, such as 20 nm or less, such as 15 nm or less, such as 10 nm or less, such as 5 nm or less, such as 2 nm or less and including light sources which emit a specific wavelength of light. In some instances, the polymerizable composition is irradiated with a narrow band light source such as a narrow wavelength LED, laser diode or a broadband light source coupled to one or more optical bandpass filters, diffraction gratings, monochromators or any combination thereof.

In certain embodiments, the light source is a stroboscopic light source and the polymerizable composition is illuminated with periodic flashes of light, such as where the polymerizable composition is irradiated at a frequency of 0.01 kHz or greater, such as 0.05 kHz or greater, such as 0.1 kHz or greater, such as 0.5 kHz or greater, such as 1 kHz or greater, such as 2.5 kHz or greater, such as 5 kHz or greater, such as 10 KHz or greater, such as 25 kHz or greater, such as 50 kHz or greater and including 100 kHz or greater. In certain instances, the polymerizable composition is irradiated with a laser, such as pulsed laser or a continuous wave laser.

In some embodiments, the polymerizable composition is in contact with the build elevator and the build surface. In some instances, methods include irradiating the polymerizable composition for 1 second or longer to bond the first polymerized region of the polymerizable composition to the build elevator, such as from 5 seconds longer, such as for 10 seconds or longer, such as for 20 seconds or longer, such as for 30 seconds or longer, such as for 1 minute or longer, such as for 5 minutes or longer and including for 10 minutes or longer.

In some embodiments, the build elevator is displaced away from the build surface after the first polymerized region of the polymerizable composition is bonded to the build elevator. In some instances, the build elevator is displaced in increments of 0.001 μm or more, such as 0.005 μm or more, such as 0.01 μm or more, such as 0.05 μm or more, such as 0.1 μm or more, such as 0.5 μm or more, such as 1 μm or more, such as 2 μm or more, such as 3 μm or more, such as 4 μm or more, such as 5 μm or more and including in increments of 10 μm or more. In certain instances, the build elevator is displaced in increments of from 0.001 μm to 20 μm, such as from 0.005 μm to 19 μm, such as from 0.01 μm to 18 μm, such as from 0.05 μm to 17 μm, such as from 0.1 μm to 16 μm, such as from 0.2 μm to 17 μm, such as from 0.3 μm to 16 μm, such as from 0.4 μm to 15 μm, such as from 0.5 μm to 14 μm, such as from 0.6 μm to 13 μm, such as from 0.7 μm to 12 μm, such as from 0.8 μm to 11 μm and including from 0.9 μm to 10 μm.

In certain instances, polymerizable composition is added to the build surface after each displacement of the build elevator away from the build surface. In some instances, the polymerizable composition is continuously added to the build surface. In other instances, the polymerizable composition is added to the build surface in discreet intervals each having a predetermined amount. In some embodiments, the polymerizable composition is selected from polycaprolactone, polyglycolic acid, polylactic acid, polylactic-co-glycolic acid, polyethylene glycol, polyethylene glycol dimethacrylate (PEGDMA), thiol-enes, anhydrides, polyacrylic acid, poly methylmethacrylate, trimethylolpropane triacrylate (TMPTA) monomer, polyvinyl alcohol, polyvinylpyrrolidone, vinyl carbonates, vinyl esters, acrylamides, hyaluronic acid, chitosan, collagen, gelatin, carboxymethylcellulose, and blends or copolymers thereof. In certain instances, one or more of the polymerizable materials includes carbon nanotubes, such as single-walled carbon nanotubes (SWCNTs) or multi-walled carbon nanotubes (MWCNTs). In some instances, the polymerizable composition has a viscosity of from 100 cP to 7000 cP, such as from 150 cP to 6500 cP, such as from 200 cP to 6000 cP, such as from 250 cP to 5500 cP, such as from 300 cP to 5000 cP, such as from 350 cP to 4500 cP, such as from 400 cP to 4000 cP, such as from 450 cP to 3500 cP and including a viscosity of from 500 cP to 3000 cP.

In some embodiments, the polymerizable composition is irradiated through the build surface. In some instances, the polymerizable composition is irradiated in the presence of a polymerization inhibitor. In certain embodiments, the polymerizable composition is continuously polymerized while displacing the build elevator away from the build surface. In certain cases, the polymerization inhibitor is oxygen and the build surface is permeable to oxygen. In certain instances, polymerizing the polymerizable composition in the presence of a polymerization inhibitor such as oxygen enables continuous (i.e., not layer-by-layer) generation of the polymeric structure with a micro-void space with a liquid “dead zone” at the interface between the build surface and the building polymeric structure having the micro-void space. In some instances, the dead zone is generated because oxygen acts as a polymerization inhibitor, passing through the oxygen-permeable build surface. Photopolymerization cannot occur in the oxygen containing “dead zone” region such that this region remains fluid, and the polymerized component in contact with the build surface so that the building polymeric structure does not physically attach to the build surface.

In some embodiments, the polymerizable composition is in contact with the build elevator and the build surface. In some instances, the method includes irradiating the polymerizable composition for a duration sufficient to bond the first polymerized region of the polymerizable composition to the build elevator. In some instances, the build elevator is displaced in increments of 0.001 μm or more, such as 0.005 μm or more, such as 0.01 μm or more, such as 0.05 μm or more, such as 0.1 μm or more, such as 0.5 μm or more, such as 1 μm or more, such as 2 μm or more, such as 3 μm or more, such as 4 μm or more, such as 5 μm or more and including in increments of 10 μm or more. In certain instances, the build elevator is displaced in increments of from 0.001 μm to 20 μm, such as from 0.005 μm to 19 μm, such as from 0.01 μm to 18 μm, such as from 0.05 μm to 17 μm, such as from 0.1 μm to 16 μm, such as from 0.2 μm to 17 μm, such as from 0.3 μm to 16 μm, such as from 0.4 μm to 15 μm, such as from 0.5 μm to 14 μm, such as from 0.6 μm to 13 μm, such as from 0.7 μm to 12 μm, such as from 0.8 μm to 11 μm and including from 0.9 μm to 10 μm.

In some instances, the methods include adding polymerizable composition to the build surface after each displacement of the build elevator away from the build surface. In some instances, methods include continuously adding polymerizable composition to the build surface. In some instances, the polymerizable composition is continuously added to the build surface by injection through a conduit.

In some instances, the polymerizable composition may be provided directly to the build plate from a liquid conduit and reservoir system. In some embodiments the carrier include one or more feed channels therein. The carrier feed channels are in fluid communication with the polymerizable composition source, for example a reservoir and associated pump. Different carrier feed channels may be in fluid communication with the same supply and operate simultaneously with one another, or different carrier feed channels may be separately controllable from one another (for example, through the provision of a pump and/or valve for each). Separately controllable feed channels may be in fluid communication with a source (e.g., reservoir) containing the same polymerizable composition, or may be in fluid communication with a reservoir containing different polymerizable compositions. Through the use of valve assemblies, different polymerizable compositions may in some embodiments be alternately fed through the same feed channel, if desired.

In some embodiments, the polymerizable composition is conveyed to the space between the build elevator and the build surface through two or more conduits, such as 3 or more, such as 4 or more, such as 5 or more, such as 6 or more, such as 7 or more, such as 8 or more, such as 9 or more and including through 10 or more different conduits. In some instances, the conduit is positioned internal to the generated polymeric structure. In other instances, the conduit is positioned external to the generated polymeric structure. In certain instances, one or more of the conduits passes through the build elevator, such as 2 or more of the conduits, such as 3 or more of the conduits and including where polymerizable composition is conveyed through 5 or more of the conduits that pass through the build elevator.

In some embodiments, methods include conveying two or more different polymerizable materials into the space between the build elevator and the build surface. In some instances, a first polymerizable material is conveyed through a first conduit into the space between the build elevator and the build surface and a second polymerizable material is conveyed through a second conduit into the space between the build elevator and the build surface. In certain embodiments, a plurality of different polymerizable materials is conveyed through a plurality of different conduits into the space between the build elevator and the build surface. For example, the number of different polymerizable materials conveyed may be 2 or more, such as 3 or more, such as 4 or more, such as 5 or more, such as 6 or more, such as 7 or more, such as 8 or more, such as 9 or more and including 10 or more. In some instances, the plurality of polymerizable materials is conveyed through 2 or more different conduits, such as 3 or more, such as 4 or more, such as 5 or more, such as 6 or more, such as 7 or more, such as 8 or more, such as 9 or more and including 10 or more different conduits.

The two or more different polymerizable materials may be conveyed into the space between the build elevator and the build surface simultaneously or in a predetermined sequential order. In some instances, two or more different polymerizable materials are conveyed into the space between the build elevator and the build surface, such as to form a blend or mixture of the two or more different polymerizable materials (i.e., a mixed resin). In other instances, two or more different polymerizable materials are conveyed sequentially into the space between the build elevator and the build surface, such as to form layers of different polymerizable materials.

The polymerizable composition may be conveyed through each conduit at a rate that varies, such as from 0.01 μL/s to 200 μL/s, such as from 0.05 μL/s to 150 μL/s, such as from 0.1 μL/s to 100 μL/s, such as from 0.5 μL/s to 90 μL/s, such as from 1 L/s to 80 μL/s, such as from 2 L/s to 70 μL/s, such as from 3 μL/s to 60 μL/s, such as from 4 μL/s to 50 L/s, such as from 5 μL/s to 40 μL/s, such as from 6 μL/s to 30 μL/s and including from 7 μL/s to 27 μL/s. In some instances, the rate for conveying the polymerizable composition is controlled by a syringe pump. In some instances, the rate may be controlled by a rate-limiting valve positioned at a proximal or distal end of the conduit. In certain embodiments, the polymerizable composition is conveyed into the space between the build elevator and the build surface at a rate sufficient to generate the polymeric structure at a rate of 0.01 mm/hr or more, such as 0.05 mm/hr or more, such as 0.1 mm/hr or more, such as 0.5 mm/hr or more, such as 1 mm/hr or more, such as 2 mm/hr or more, such as 3 mm/hr or more, such as 4 mm/hr or more, such as 5 mm/hr or more, such as 6 mm/hr or more, such as 7 mm/hr or more, such as 8 mm/hr or more, such as 9 mm/hr or more, such as 10 mm/hr or more, such as 15 mm/hr or more, such as 20 mm/hr or more, such as 25 mm/hr or more, such as 50 mm/hr or more, such as 75 mm/hr or more, such as 100 mm/hr or more, such as 150 mm/hr or more and including conveying the polymerizable composition through one or more conduits into the space between the build elevator and the build surface at a rate sufficient to generate the polymeric structure at a rate of 250 mm/hr or more. For example, the polymerizable material may be conveyed through one or more conduits at a rate sufficient to generate the polymeric structure at a rate of 1 mm/hr to 250 mm/hr, such as from 2 mm/hr to 225 mm/hr, such as from 3 mm/hr to 200 mm/hr, such as from 4 mm/hr to 175 mm/hr, such as from 5 mm/hr to 150 mm/hr and including from 10 mm/hr to 125 mm/hr.

In some embodiments, methods providing the polymerizable composition to a liquid interface polymerization module through injection continuous liquid interface production such as that described in International Patent Application No. PCT/US23/15406 filed on Mar. 16, 2023, the disclosure of which is herein incorporated by reference. In certain embodiments, the polymerizable composition is polymerized using a liquid interface polymerization module that is a continuous liquid interface production (CLIP) system such as that described in International Patent Publication No. WO 2014/126837; U.S. Patent Publication Nos. 2018/0064920; 2017/0095972; 2021/0246252 and U.S. Pat. Nos. 10,155,882; 10,792,857, the disclosures of which are herein incorporated by reference.

In some instances, the micro-void space generated within the polymeric structure includes one or more microchannels. In some instances, one or more of the microchannels includes one or more bifurcations, such as 2 or more bifurcations, such as 3 or more, such as 4 or more, such as 5 or more and including 10 or more different bifurcations. In some instances, the microchannels extend through the polymeric structure. In some instances, the microchannels are fluidically interconnected. In some instances, the polymeric structure has a single network of fluidically interconnected microchannel networks. In other instances, the polymeric structure has a plurality of fluidically interconnected microchannel networks.

In practicing methods of the present disclosure according to some embodiments, an amount of the polymerizable composition is conveyed through the generated micro-void space in a manner sufficient to displace any material (e.g., residual or trapped polymerized resin) in the micro-void space. In embodiments, the polymerizable composition used to displace material from the micro-void space may be the same polymerizable material used to form the polymeric structure or may be a different polymerizable material as desired. In some instances, the polymerizable composition conveyed through the micro-void space is non-reactive when injected through the micro-void space. By “non-reactive” is meant that the polymerizable composition conveyed through the micro-void space does not react with the formed polymeric structure and does not polymerize therein (such as to obstruct the micro-void space). In some instances, the polymerizable composition is injected into the micro-void space, such as where the micro-void space is a microchannel within the forming polymeric structure. The polymerizable composition may be conveyed through the generated micro-void space at a rate sufficient to displace the material in the micro-void space, such as a rate of 0.01 μL/s or more, such as 0.05 μL/s or more, such as 0.1 μL/s or more, such as 0.5 μL/s or more, such as 1 μL/s or more, such as 2 μL/s or more, such as 3 μL/s or more, such as 4 μL/s or more, such as 5 μL/s or more, such as 6 μL/s or more, such as 7 μL/s or more, such as 8 μL/s or more, such as 9 μL/s or more, such as 10 μL/s or more, such as 15 L/s or more, such as 20 μL/s or more, such as 25 L/s or more, such as 50 μL/s or more, such as 75 μL/s or more, such as 100 L/s or more and including at a rate of 250 μL/s or more. For example, the polymerizable composition may be conveyed through the generated micro-void space at a rate of from 0.01 L/s to 200 μL/s, such as from 0.05 μL/s to 150 μL/s, such as from 0.1 μL/s to 100 μL/s, such as from 0.5 μL/s to 90 μL/s, such as from 1 μL/s to 80 μL/s, such as from 2 μL/s to 70 μL/s, such as from 3 μL/s to 60 μL/s, such as from 4 μL/s to 50 μL/s, such as from 5 μL/s to 40 L/s, such as from 6 μL/s to 30 μL/s and including from 7 μL/s to 27 μL/s. In certain instances, conveying the polymerizable composition is sufficient to flush out trapped or residual resin in the micro-void space in order to preserve the negative space and eliminate print-through of the polymeric structure.

In some embodiments, the polymerizable composition is conveyed through the generated micro-void space at predetermined intervals while generating that polymeric structure (e.g., while displacing the build elevator away from the build surface when generating the polymeric structure). In some instances, the polymerizable composition is conveyed through the micro-void space to displace any material (e.g., trapped polymerized resin) in the micro-void space every 1 second or more, such as every 5 seconds or more, such as every 10 seconds or more, such as every 15 seconds or more, such as every 30 seconds or more, such as every 1 minute or more, such as every 5 minutes or more, such as every 10 minutes or more and including every 30 minutes or more. In some instances, the polymerizable composition is continuously conveyed through the generated micro-void space into the space between the build elevator and the build surface of the liquid interface production module. In some instances, the non-reactive composition is continuously conveyed (e.g., through a conduit) into the micro-void space while displacing the build elevator away from the build surface when generating the polymeric structure.

In some embodiments, methods include contacting the generated micro-void space with a non-polymerizable composition. In practicing the subject methods, in some instances the non-polymerizable composition is continuously contacted with the micro-void space while generating the polymeric structure. In some embodiments, methods include filling at least a part of the void volume of the micro-void space, such as 5% or more, such as 10% or more, such as 15% or more, such as 20% or more, such as 25% or more, such as 50% or more, such as 75% or more and including 90% or more of the void volume of the micro-void space with the non-polymerizable composition. In some embodiments, methods include filling the entire void volume of the micro-void space with the non-polymerizable composition. In certain instances, the non-polymerizable composition is non-reactive with the polymerizable composition of the polymeric structure. In some instances, the non-polymerizable composition fills one or more microchannel within the polymeric structure, such as where the non-polymerizable composition fills one or more microchannels which extend through the polymeric structure. In some instances, the polymeric structure includes a plurality of micro-void spaces. In some instances, the non-polymerizable composition is a composition selected from water, a Newtonian liquid, a shear thinning liquid, a shear thickening liquid, a magnetorheological liquid, an electric field responsive liquid and a gas.

In some instances, the methods include adding non-polymerizable composition to the build surface to fill the generated micro-void spaces after each displacement of the build elevator away from the build surface. In some instances, methods include continuously adding non-polymerizable composition to the build surface to fill the generated micro-void spaces. In some instances, the non-polymerizable composition is continuously added to the build surface to fill the generated micro-void spaces by injection through a conduit.

In some instances, the non-polymerizable composition may be provided directly to the build plate from a liquid conduit and reservoir system. In some embodiments the carrier include one or more feed channels therein. The carrier feed channels are in fluid communication with the non-polymerizable composition supply, for example a reservoir and associated pump. Different carrier feed channels may be in fluid communication with the same supply and operate simultaneously with one another, or different carrier feed channels may be separately controllable from one another (for example, through the provision of a pump and/or valve for each). Separately controllable feed channels may be in fluid communication with a reservoir containing the same polymerizable composition, or may be in fluid communication with a reservoir containing different polymerizable compositions. Through the use of valve assemblies, different polymerizable compositions may in some embodiments be alternately fed through the same feed channel, if desired.

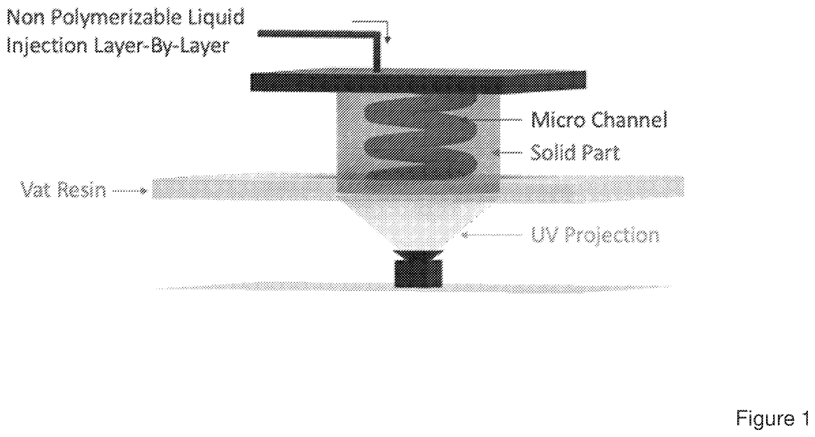

FIG. 1 depicts injecting a non-polymerizable composition into a generated micro-void space, such as a microchannel, according to certain embodiments. As shown in FIG. 1, the polymeric structure is generated layer-by-layer by irradiating the polymerizable composition between the build elevator and the build surface. A micro-void space, here a microchannel is formed within the polymeric structure. During formation of the microchannel at each layer, the non-polymerizable composition is injected through a conduit through the build elevator into the forming micro-void space. The non-polymerizable composition in this embodiment is sufficient to prevent polymerization of excess polymerizable material in the forming micro-void space. Removal of the non-polymerizable composition is sufficient to generate a resolved micro-void space, such as the depicted microchannel through the polymeric structure. As described above, in some instances, additional polymerizable material (e.g., fresh resin used to form the polymeric structure) can instead be injected through the forming microchannel at each layer to flush out residual or trapped polymerized material.

### Systems for Making a Polymeric Structure Having a Micro-Void Space

Aspects of the disclosure also include systems for making a polymeric structure have a micro-void space. Systems according to certain embodiments include a light source and a light interface polymerization module having a build elevator and a build surface configured for generating a polymeric structure having a resolved micro-void space therein from a polymerizable composition positioned therebetween.

In some embodiments, systems include a light source. In some embodiments, the light source is a broadband light source, emitting light having a broad range of wavelengths, such as for example, spanning 50 nm or more, such as 100 nm or more, such as 150 nm or more, such as 200 nm or more, such as 250 nm or more, such as 300 nm or more, such as 350 nm or more, such as 400 nm or more and including spanning 500 nm or more. For example, one suitable broadband light source emits light having wavelengths from 200 nm to 1500 nm. Another example of a suitable broadband light source includes a light source that emits light having wavelengths from 400 nm to 1000 nm. Any convenient broadband light source protocol may be employed, such as a halogen lamp, deuterium arc lamp, xenon arc lamp, stabilized fiber-coupled broadband light source, a broadband LED with continuous spectrum, superluminescent emitting diode, semiconductor light emitting diode, wide spectrum LED white light source, a multi-LED integrated white light source, among other broadband light sources or any combination thereof.

In some embodiments, the light source is a narrow band light source emitting a particular wavelength or a narrow range of wavelengths. In some instances, the narrow band light sources emit light having a narrow range of wavelengths, such as for example, 50 nm or less, such as 40 nm or less, such as 30 nm or less, such as 25 nm or less, such as 20 nm or less, such as 15 nm or less, such as 10 nm or less, such as 5 nm or less, such as 2 nm or less and including light sources which emit a specific wavelength of light (i.e., monochromatic light). Any convenient narrow band light source protocol may be employed, such as a narrow wavelength LED, laser diode or a broadband light source coupled to one or more optical bandpass filters, diffraction gratings, monochromators or any combination thereof. The subject systems may include one or more light sources, as desired, such as two or more light sources, such as three or more light sources, such as four or more light sources, such as five or more light sources and including ten or more light sources. The light source may include a combination of types of light sources, for example, where two lights sources are employed, a first light source may be a broadband white light source (e.g., broadband white light LED) and second light source may be a broadband near-infrared light source (e.g., broadband near-IR LED). In other instances, where two light sources are employed, a first light source may be a broadband white light source (e.g., broadband white light LED) and the second light source may be a narrow spectra light source (e.g., a narrow band visible light or near-IR LED). In yet other instances, the light source is an plurality of narrow band light sources each emitting specific wavelengths, such as an array of two or more LEDs, such as an array of three or more LEDs, such as an array of five or more LEDs, including an array of ten or more LEDs.

In certain embodiments, the light source is a stroboscopic light source where the polymerizable composition is illuminated with periodic flashes of light. Depending on the light source (e.g., flash lamp, pulsed laser) the frequency of light strobe may vary, and may be 0.01 kHz or greater, such as 0.05 kHz or greater, such as 0.1 kHz or greater, such as 0.5 kHz or greater, such as 1 kHz or greater, such as 2.5 kHz or greater, such as 5 kHz or greater, such as 10 kHz or greater, such as 25 kHz or greater, such as 50 kHz or greater and including 100 KHz or greater. In these embodiments, the strobe light may be operably coupled to a processor having a frequency generator which regulates strobe frequency. In some instances, the frequency generator of the strobe light is operably coupled to the projection monitoring component of the micro-digital light projection system such that the frequency of the strobe light is synchronized with the frequency of image capture on the build surface of the light interface polymerization module. In certain instances, suitable strobe light sources and frequency controllers include, but are not limited to those described in U.S. Pat. Nos. 5,700,692 and 6,372,506, the disclosures of which are herein incorporated by reference.

In some embodiments, the light source includes one or more lasers. Lasers of interest may include pulsed lasers or continuous wave lasers. The type and number of lasers used in the subject methods may vary and may be a gas laser, such as a helium-neon laser, argon laser, krypton laser, xenon laser, nitrogen laser, CO₂ laser, CO laser, argon-fluorine (ArF) excimer laser, krypton-fluorine (KrF) excimer laser, xenon chlorine (XeCI) excimer laser or xenon-fluorine (XeF) excimer laser or a combination thereof. In others instances, the light beam generator includes a dye laser, such as a stilbene, coumarin or rhodamine laser. In yet other instances, the light beam generator includes a metal-vapor laser, such as a helium-cadmium (HeCd) laser, helium-mercury (HeHg) laser, helium-selenium (HeSe) laser, helium-silver (HeAg) laser, strontium laser, neon-copper (NeCu) laser, copper laser or gold laser and combinations thereof. In still other instances, the light beam generator includes a solid-state laser, such as a ruby laser, an Nd:YAG laser, NdCrYAG laser, Er:YAG laser, Nd:YLF laser, Nd:YVO₄ laser, Nd:YCa₄O(BO₃)₃ laser, Nd:YCOB laser, titanium sapphire laser, thulium YAG laser, ytterbium YAG laser, ytterbium₂O₃ laser or cerium doped lasers and combinations thereof. In still other instances, the light source includes a semiconductor diode laser, optically pumped semiconductor laser (OPSL), or a frequency doubled- or frequency tripled implementation of any of the above mentioned lasers.

In some embodiments, the light source includes one or more tube lenses that are configured with adjustable focal lengths. In some instances, the tube lens is a telecentric lens. In certain instances, the tube lens is configured for widefield imaging. In some instances, the tube lens has an adjustable focal length which ranges from 10 mm to 1000 mm, such as from 20 mm to 900 mm, such as from 30 mm to 800 mm, such as from 40 mm to 700 mm, such as from 50 mm to 600 mm, such as from 60 mm to 500 mm, such as from 70 mm to 400 mm, such as from 80 mm to 300 mm and including an adjustable focal length of from 100 mm to 200 mm.

In some embodiments, the light source includes one or more projection lenses, such as 2 or more projection lenses, such as 3 or more projection lenses, such as 4 or more projection lenses and including 5 or more projection lenses. In some instances, the projection lenses provide for magnification of 2-fold or more, such as 3-fold or more, such as 4-fold or more, such as 5-fold or more, such as 6-fold or more, such as 7-fold or more, such as 8-fold or more, such as 9-fold or more and including 10-fold or more magnification. In some instances, the projection lenses provide for de-magnification having a magnification ratio ranging from 0.1 to 0.95, such as a magnification ratio of from 0.2 to 0.9, such as a magnification ratio of from 0.3 to 0.85, such as a magnification ratio of from 0.35 to 0.8, such as a magnification ratio of from 0.5 to 0.75 and including a magnification ratio of from 0.55 to 0.7, for example a magnification ratio of 0.6.

In some embodiments, the light source includes one or more beamsplitters. The beamsplitter may be any an optical component that is configured to propagate a beam of light along two or more different and spatially separated optical paths, such that a predetermined portion of the light is propagated along each optical path. The beamsplitter may be any convenient beamsplitting protocol such as with triangular prism, slivered mirror prisms, dichroic mirror prisms, among other types of beamsplitters. The beamsplitter may be formed from any suitable material so long as the beamsplitter is capable of propagating the desired amount and wavelengths of light along each optical path. For example, beamsplitters of interest may be formed from glass (e.g., N-SF10, N-SF11, N-SF57, N-BK7, N-LAK21 or N-LAF35 glass), silica (e.g., fused silica), quartz, crystal (e.g., CaF₂ crystal), zinc selenide (ZnSe), F₂, germanium (Ge) titanate (e.g., S-TIH11), borosilicate (e.g., BK7). In certain embodiments, the beamsplitter is formed from a polymeric material, such as, but not limited to, polycarbonates, polyvinyl chloride (PVC), polyurethanes, polyethers, polyamides, polyimides, or copolymers of these thermoplastics, such as PETG (glycol-modified polyethylene terephthalate), among other polymeric plastic materials. In certain embodiments, the beamsplitter is formed from a polyester, where polyesters of interest may include, but are not limited to, poly(alkylene terephthalates) such as poly(ethylene terephthalate) (PET), bottle-grade PET (a copolymer made based on monoethylene glycol, terephthalic acid, and other comonomers such as isophthalic acid, cyclohexene dimethanol, etc.), poly(butylene terephthalate) (PBT), and poly(hexamethylene terephthalate); poly(alkylene adipates) such as poly(ethylene adipate), poly(1,4-butylene adipate), and poly(hexamethylene adipate); poly(alkylene suberates) such as poly(ethylene suberate); poly(alkylene sebacates) such as poly(ethylene sebacate); poly(ε-caprolactone) and poly(β-propiolactone); poly(alkylene isophthalates) such as poly(ethylene isophthalate); poly(alkylene 2,6-naphthalene-dicarboxylates) such as poly(ethylene 2,6-naphthalene-dicarboxylate); poly(alkylene sulfonyl-4,4′-dibenzoates) such as poly(ethylene sulfonyl-4,4′-dibenzoate); poly(p-phenylene alkylene dicarboxylates) such as poly(p-phenylene ethylene dicarboxylates); poly(trans-1,4-cyclohexanediyl alkylene dicarboxylates) such as poly(trans-1,4-cyclohexanediyl ethylene dicarboxylate); poly(1,4-cyclohexane-dimethylene alkylene dicarboxylates) such as poly(1,4-cyclohexane-dimethylene ethylene dicarboxylate); poly([2.2.2]-bicyclooctane-1,4-dimethylene alkylene dicarboxylates) such as poly([2.2.2]-bicyclooctane-1,4-dimethylene ethylene dicarboxylate); lactic acid polymers and copolymers such as(S)-polylactide, (R,S)-polylactide, poly(tetramethylglycolide), and poly(lactide-co-glycolide); and polycarbonates of bisphenol A, 3,3′-dimethylbisphenol A, 3,3′,5,5′-tetrachlorobisphenol A, 3,3′,5,5′-tetramethylbisphenol A; polyamides such as poly(p-phenylene terephthalamide); polyethylene Terephthalate (e.g., Mylar™ Polyethylene Terephthalate), combinations thereof, and the like.

In embodiments, the micro-digital light projection system includes a light projection monitoring component having a photodetector. Photodetectors may be any convenient light detecting protocol, including but not limited to photosensors or photodetectors, such as active-pixel sensors (APSs), avalanche photodiodes (APDs), quadrant photodiodes, image sensors, charge-coupled devices (CCDs), intensified charge-coupled devices (ICCDs), light emitting diodes, photon counters, bolometers, pyroelectric detectors, photoresistors, photovoltaic cells, photodiodes, photomultiplier tubes, phototransistors, quantum dot photoconductors or photodiodes and combinations thereof, among other photodetectors. In certain embodiments, the photodetector is a photomultiplier tube, such as a photomultiplier tube having an active detecting surface area of each region that ranges from 0.01 cm² to 10 cm², such as from 0.05 cm² to 9 cm², such as from, such as from 0.1 cm² to 8 cm², such as from 0.5 cm² to 7 cm² and including from 1 cm² to 5 cm².

In certain embodiments, the light projection monitoring component includes one or more photodetectors that are optically coupled to a slit. Depending on the size of the active detecting surface of the photodetector, slits according to certain instances have a rectangular (or other polygonal shape) opening having a width of from 0.01 mm to 2 mm, such as from 0.1 mm to 1.9 mm, such as from 0.2 mm to 1.8 mm, such as from 0.3 mm to 1.7 mm, such as from 0.4 mm to 1.6 mm, and including a width of from 0.5 mm to 1.5 mm and a length of from 0.01 mm to 2 mm, such as from 0.1 mm to 1.9 mm, such as from 0.2 mm to 1.8 mm, such as from 0.3 mm to 1.7 mm, such as from 0.4 mm to 1.6 mm, and including a length of from 0.5 mm to 1.5 mm. In certain instances, the width of the slit is 1 mm or less, such as 0.9 mm or less, such as 0.8 mm or less, such as 0.7 mm or less, such as 0.6 mm or less, such as 0.5 mm or less and including a width that is 0.4 mm or less. In certain instances, the light detection system includes a photodetector that is optically coupled to a slit having a plurality of openings, such as a slit having 2 or more openings, such as 3 or more openings, such as 4 or more openings, such as 5 or more openings, such as 6 or more openings, such as 7 or more openings, such as 8 or more openings, such as 9 or more openings and including a slit having 10 or more openings.

Light may be measured by the photodetector at one or more wavelengths, such as at 2 or more wavelengths, such as at 5 or more different wavelengths, such as at 10 or more different wavelengths, such as at 25 or more different wavelengths, such as at 50 or more different wavelengths, such as at 100 or more different wavelengths, such as at 200 or more different wavelengths, such as at 300 or more different wavelengths and including measuring light at 400 or more different wavelengths. Light may be measured continuously or in discrete intervals. In some instances, detectors of interest are configured to take measurements of the light continuously. In other instances, detectors of interest are configured to take measurements in discrete intervals, such as measuring light every 0.001 millisecond, every 0.01 millisecond, every 0.1 millisecond, every 1 millisecond, every 10 milliseconds, every 100 milliseconds and including every 1000 milliseconds, or some other interval.

In certain embodiments, the micro-digital light projection system is a digital light processing (DLP) system having a digital micromirror device such as that described in U.S. Patent Publication Nos. 2017/0095972; 2022/0250313; 2022/0048242 and U.S. Pat. Nos. 11,358,342; 11,141,910, the disclosures of which are herein incorporated by reference.

In some embodiments, systems include a processor having memory operably coupled to the processor where the memory includes instructions stored thereon, which when executed by the processor, cause the processor to a) irradiate a polymerizable composition positioned between a build elevator and a build surface to generate a polymerizable composition having a polymerized region of the polymerizable composition with a micro-void space in contact with the build elevator and a non-polymerized region of the polymerizable composition in contact with the build surface; b) displace the build elevator away from the build surface; c) contact the generated micro-void space with a non-reactive composition; and d) repeat steps a)-c) in a manner sufficient to generate a polymeric structure having a resolved micro-void space. In some instances, systems are configured to generate a polymeric structure having a micro-void space that includes a non-polymerizable composition positioned therein. These steps are repeated in a manner sufficient to generate a polymeric structure having a micro-void space having a non-polymerizable composition positioned therein. For example, the steps may be repeated 2 or more times, such as 3 or more times, such as 4 or more times, such as 5 or more times, such as 10 or more times, such as 20 or more times, such as 30 or more times, such as 40 or more times, such as 50 or more times, such as 100 or more times, such as 250 or more times, such as 500 or more times and including 1000 or more times.

In some embodiments, the memory includes instructions to irradiate the polymerizable composition for a duration sufficient to bond the first polymerized region of the polymerizable composition to the build elevator. In some instances, the memory includes instructions to irradiate the polymerizable composition for 1 second or longer to bond the first polymerized region of the polymerizable composition to the build elevator, such as from 5 seconds longer, such as for 10 seconds or longer, such as for 20 seconds or longer, such as for 30 seconds or longer, such as for 1 minute or longer, such as for 5 minutes or longer and including for 10 minutes or longer.

In some embodiments, the memory includes instructions to displace the build elevator in predetermined increments which builds the polymeric structure. In some instances, the memory includes instructions to displace the build elevator in increments of 0.001 μm or more, such as 0.005 μm or more, such as 0.01 μm or more, such as 0.05 μm or more, such as 0.1 μm or more, such as 0.5 μm or more, such as 1 μm or more, such as 2 μm or more, such as 3 μm or more, such as 4 μm or more, such as 5 μm or more and including in increments of 10 μm or more. In certain instances, the memory includes instructions to displace the build elevator in increments of from 0.001 μm to 20 μm, such as from 0.005 μm to 19 μm, such as from 0.01 μm to 18 μm, such as from 0.05 μm to 17 μm, such as from 0.1 μm to 16 μm, such as from 0.2 μm to 17 μm, such as from 0.3 μm to 16 μm, such as from 0.4 μm to 15 μm, such as from 0.5 μm to 14 μm, such as from 0.6 μm to 13 μm, such as from 0.7 μm to 12 μm, such as from 0.8 μm to 11 μm and including from 0.9 μm to 10 μm.

In certain instances, the memory includes instructions for adding polymerizable composition to the build surface after each displacement of the build elevator away from the build surface. In some instances, the memory includes instructions for continuously adding the polymerizable composition to the build surface. In other instances, the memory include instructions for continuously adding polymerizable composition to the build surface in discreet intervals each having a predetermined amount. In some embodiments, the polymerizable composition is selected from polycaprolactone, polyglycolic acid, polylactic acid, polylactic-co-glycolic acid, polyethylene glycol, polyethylene glycol dimethacrylate (PEGDMA), thiol-enes, anhydrides, polyacrylic acid, poly methylmethacrylate, trimethylolpropane triacrylate (TMPTA) monomer, polyvinyl alcohol, polyvinylpyrrolidone, vinyl carbonates, vinyl esters, acrylamides, hyaluronic acid, chitosan, collagen, gelatin, carboxymethylcellulose, and blends or copolymers thereof. In certain instances, one or more of the polymerizable materials includes carbon nanotubes, such as single-walled carbon nanotubes (SWCNTs) or multi-walled carbon nanotubes (MWCNTs). In some instances, the polymerizable composition has a viscosity of from 100 cP to 7000 cP, such as from 150 cP to 6500 cP, such as from 200 cP to 6000 cP, such as from 250 cP to 5500 cP, such as from 300 cP to 5000 cP, such as from 350 cP to 4500 cP, such as from 400 cP to 4000 cP, such as from 450 cP to 3500 cP and including a viscosity of from 500 cP to 3000 cP.

In some instances, the polymerizable composition may be provided directly to the build plate from a liquid conduit and reservoir system. In some embodiments the carrier includes one or more feed channels therein. The carrier feed channels are in fluid communication with the polymerizable composition source, for example a reservoir and associated pump. Different carrier feed channels may be in fluid communication with the same supply and operate simultaneously with one another, or different carrier feed channels may be separately controllable from one another (for example, through the provision of a pump and/or valve for each). Separately controllable feed channels may be in fluid communication with a source (e.g., reservoir) containing the same polymerizable composition, or may be in fluid communication with a reservoir containing different polymerizable compositions. Through the use of valve assemblies, different polymerizable compositions may in some embodiments be alternately fed through the same feed channel, if desired.

In some embodiments, the system includes two or more conduits for conveying polymerizable composition to the space between the build elevator and the build surface, such as 3 or more, such as 4 or more, such as 5 or more, such as 6 or more, such as 7 or more, such as 8 or more, such as 9 or more and including through 10 or more different conduits. In some instances, the conduit is positioned internal to the generated polymeric structure. In other instances, the conduit is positioned external to the generated polymeric structure. In certain instances, one or more of the conduits passes through the build elevator, such as 2 or more of the conduits, such as 3 or more of the conduits and including where polymerizable composition is conveyed through 5 or more of the conduits that pass through the build elevator.

In some embodiments, systems are configured to convey two or more different polymerizable materials into the space between the build elevator and the build surface. In some instances, the system is configured to convey a first polymerizable material through a first conduit into the space between the build elevator and the build surface and to convey a second polymerizable material through a second conduit into the space between the build elevator and the build surface. In certain embodiments, the system is configured to convey a plurality of different polymerizable materials through a plurality of different conduits into the space between the build elevator and the build surface. For example, the number of different polymerizable materials conveyed may be 2 or more, such as 3 or more, such as 4 or more, such as 5 or more, such as 6 or more, such as 7 or more, such as 8 or more, such as 9 or more and including 10 or more. In some instances, the system is configured to convey a plurality of polymerizable materials through 2 or more different conduits, such as 3 or more, such as 4 or more, such as 5 or more, such as 6 or more, such as 7 or more, such as 8 or more, such as 9 or more and including 10 or more different conduits.

In some embodiments, the system is configured to convey the two or more different polymerizable materials into the space between the build elevator and the build surface simultaneously or in a predetermined sequential order. In some instances, systems are configured to convey two or more different polymerizable materials into the space between the build elevator and the build surface, such as to form a blend or mixture of the two or more different polymerizable materials (i.e., a mixed resin). In other instances, systems are configured to convey two or more different polymerizable materials sequentially into the space between the build elevator and the build surface, such as to form layers of different polymerizable materials.

In some instances, system are configured to convey the polymerizable composition through each conduit at a rate that varies, such as from 0.01 μL/s to 200 μL/s, such as from 0.05 μL/s to 150 μL/s, such as from 0.1 μL/s to 100 μL/s, such as from 0.5 μL/s to 90 μL/s, such as from 1 L/s to 80 μL/s, such as from 2 μL/s to 70 μL/s, such as from 3 μL/s to 60 μL/s, such as from 4 μL/s to 50 μL/s, such as from 5 μL/s to 40 μL/s, such as from 6 μL/s to 30 μL/s and including from 7 μL/s to 27 μL/s. In some instances, the rate for conveying the polymerizable composition is controlled by the syringe pump. In some instances, the conduit includes a rate-limiting valve at a proximal or distal end to control the rate of conveying the polymerizable composition. In certain embodiments, systems are configured to convey the polymerizable composition into the space between the build elevator and the build surface at a rate sufficient to generate the polymeric structure at a rate of 0.01 mm/hr or more, such as 0.05 mm/hr or more, such as 0.1 mm/hr or more, such as 0.5 mm/hr or more, such as 1 mm/hr or more, such as 2 mm/hr or more, such as 3 mm/hr or more, such as 4 mm/hr or more, such as 5 mm/hr or more, such as 6 mm/hr or more, such as 7 mm/hr or more, such as 8 mm/hr or more, such as 9 mm/hr or more, such as 10 mm/hr or more, such as 15 mm/hr or more, such as 20 mm/hr or more, such as 25 mm/hr or more, such as 50 mm/hr or more, such as 75 mm/hr or more, such as 100 mm/hr or more, such as 150 mm/hr or more and including where the system is configured to convey the polymerizable composition through one or more conduits into the space between the build elevator and the build surface at a rate sufficient to generate the polymeric structure at a rate of 250 mm/hr or more. For example, the polymerizable material may be conveyed through one or more conduits at a rate sufficient to generate the polymeric structure at a rate of 1 mm/hr to 250 mm/hr, such as from 2 mm/hr to 225 mm/hr, such as from 3 mm/hr to 200 mm/hr, such as from 4 mm/hr to 175 mm/hr, such as from 5 mm/hr to 150 mm/hr and including from 10 mm/hr to 125 mm/hr.

In some embodiments, systems include a source of the polymerizable composition in communication with the build region to add polymerizable composition to the build surface. In some instances, source is configured to add polymerizable composition to the build region through a conduit, such as by injection of the polymerizable composition through the conduit. In certain embodiments, systems of interest include a light interface polymerization module having an injection system for providing polymerizable composition to the build surface as described in International Patent Application No. PCT/US23/15406 filed on Mar. 16, 2023, the disclosure of which is herein incorporated by reference. In certain embodiments, liquid interface polymerization modules that is a continuous liquid interface production (CLIP) system include those described in International Patent Publication No. WO 2014/126837; U.S. Patent Publication Nos. 2018/0064920; 2017/0095972; 2021/0246252 and U.S. Pat. Nos. 10,155,882; 10,792,857, the disclosures of which are herein incorporated by reference.

In some embodiments, systems include a processor having memory operably coupled to the processor where the memory includes instructions stored thereon for contacting the generated micro-void space with an amount of the polymerizable composition in a manner sufficient to displace polymerized material in the micro-void space. In embodiments, the polymerizable composition used to displace material from the micro-void space may be the same polymerizable material used to form the polymeric structure or may be a different polymerizable material as desired. In some instances, the polymerizable composition conveyed through the micro-void space is non-reactive when injected through the micro-void space.

In some instances, the memory includes instructions for injecting the polymerizable composition into the micro-void space, such as where the micro-void space is a microchannel formed within the polymeric structure. In some instances, the memory includes instructions for conveying the polymerizable composition m through the generated micro-void space at a rate sufficient to displace the material in the micro-void space, such as a rate of 0.01 μL/s or more, such as 0.05 μL/s or more, such as 0.1 μL/s or more, such as 0.5 μL/s or more, such as 1 μL/s or more, such as 2 μL/s or more, such as 3 μL/s or more, such as 4 μL/s or more, such as 5 μL/s or more, such as 6 μL/s or more, such as 7 μL/s or more, such as 8 μL/s or more, such as 9 μL/s or more, such as 10 μL/s or more, such as 15 μL/s or more, such as 20 μL/s or more, such as 25 L/s or more, such as 50 μL/s or more, such as 75 μL/s or more, such as 100 μL/s or more and including at a rate of 250 μL/s or more. In certain instances, the memory includes instructions for conveying the polymerizable composition through the generated micro-void space at a rate of from 0.01 μL/s to 200 μL/s, such as from 0.05 μL/s to 150 μL/s, such as from 0.1 L/s to 100 μL/s, such as from 0.5 μL/s to 90 μL/s, such as from 1 μL/s to 80 μL/s, such as from 2 μL/s to 70 μL/s, such as from 3 μL/s to 60 μL/s, such as from 4 μL/s to 50 μL/s, such as from 5 μL/s to 40 L/s, such as from 6 μL/s to 30 μL/s and including from 7 L/s to 27 L/s. In certain instances, the memory includes instructions for conveying polymerizable composition that is sufficient to flush out trapped resin in the micro-void space in order to preserve the negative space and eliminate print-through of the polymeric structure.

In some embodiments, the memory includes instructions for conveying the polymerizable composition through the generated micro-void space at predetermined intervals while generating that polymeric structure (e.g., while displacing the build elevator away from the build surface when generating the polymeric structure). In some instances, the memory includes instructions for conveying the polymerizable composition through the micro-void space to displace any material (e.g., trapped polymerized resin) in the micro-void space every 1 second or more, such as every 5 seconds or more, such as every 10 seconds or more, such as every 15 seconds or more, such as every 30 seconds or more, such as every 1 minute or more, such as every 5 minutes or more, such as every 10 minutes or more and including every 30 minutes or more. In some instances, the memory includes instructions for conveying the polymerizable composition continuously through the generated micro-void space into the space between the build elevator and the build surface of the liquid interface production module. In some instances, the memory includes instructions for conveying the non-reactive composition continuously (e.g., through a conduit) into the micro-void space while displacing the build elevator away from the build surface when generating the polymeric structure.

In some embodiments, the memory includes instructions for contacting the generated micro-void space with a non-polymerizable composition. In some instances, the memory includes instructions for continuously contacting the non-polymerizable composition with the micro-void space while generating the polymeric structure. In some embodiments, the system further includes a source of the non-polymerizable composition that is operably coupled to the light interface polymerization module such that the non-polymerizable composition can be continuously contacted or at predetermined intervals with the micro-void space while generating the polymeric structure. In some instances, the system includes a source of non-polymerizable composition selected from water, a Newtonian liquid, a shear thinning liquid, a shear thickening liquid, a magnetorheological liquid, an electric field responsive liquid and a gas.

In some instances, the memory includes instructions for adding non-polymerizable composition to the build surface to fill the generated micro-void spaces after each displacement of the build elevator away from the build surface. In some instances, the memory includes instructions for continuously adding non-polymerizable composition to the build surface to fill the generated micro-void spaces. In some instances, the non-polymerizable composition is continuously added to the build surface to fill the generated micro-void spaces by injection through a conduit. In certain embodiments, the memory includes instructions for removing the non-polymerizable composition from the generated micro-void space of the polymeric structure.

In some instances, the memory includes instructions for filling at least a part of the void volume of the micro-void space, such as 5% or more, such as 10% or more, such as 15% or more, such as 20% or more, such as 25% or more, such as 50% or more, such as 75% or more and including 90% or more of the void volume of the micro-void space with the non-polymerizable composition, such as for filling the entire void volume of the micro-void space with the non-polymerizable composition. In some instances, system are configured to convey the non-polymerizable composition to fill at least a part of the void volume of the micro-void space through a conduit at a rate that varies, such as from 0.01 μL/s to 200 μL/s, such as from 0.05 L/s to 150 μL/s, such as from 0.1 μL/s to 100 μL/s, such as from 0.5 μL/s to 90 μL/s, such as from 1 μL/s to 80 μL/s, such as from 2 μL/s to 70 μL/s, such as from 3 μL/s to 60 μL/s, such as from 4 μL/s to 50 μL/s, such as from 5 μL/s to 40 μL/s, such as from 6 μL/s to 30 μL/s and including from 7 μL/s to 27 μL/s. In some instances, the rate for conveying the non-polymerizable composition to fill at least a part of the void volume of the micro-void space is controlled by the syringe pump.

In some embodiments, systems also include a source of the polymerizable composition. In some instances, the source is configured to continuously deliver polymerizable composition to the build surface. In some instances, the system is configured to add polymerizable composition to the build surface after each displacement of the build elevator away from the build surface. In some embodiments, the polymerizable composition is selected from polycaprolactone, polyglycolic acid, polylactic acid, polylactic-co-glycolic acid, polyethylene glycol, polyethylene glycol dimethacrylate (PEGDMA), thiol-enes, anhydrides, polyacrylic acid, poly methylmethacrylate, trimethylolpropane triacrylate (TMPTA) monomer, polyvinyl alcohol, polyvinylpyrrolidone, vinyl carbonates, vinyl esters, acrylamides, hyaluronic acid, chitosan, collagen, gelatin, carboxymethylcellulose, and blends or copolymers thereof. In certain instances, one or more of the polymerizable materials includes carbon nanotubes, such as single-walled carbon nanotubes (SWCNTs) or multi-walled carbon nanotubes (MWCNTs). In some instances, the polymerizable composition conveyed through the conduit has a viscosity of from 100 cP to 7000 cP, such as from 150 cP to 6500 cP, such as from 200 cP to 6000 cP, such as from 250 cP to 5500 cP, such as from 300 cP to 5000 cP, such as from 350 cP to 4500 cP, such as from 400 cP to 4000 cP, such as from 450 cP to 3500 cP and including a viscosity of from 500 cP to 3000 cP.

In some embodiments, the light source is configured to irradiate through the build surface. In some instances, at least a part of the build surface is permeable to a polymerization inhibitor, such as where the polymerization inhibitor is oxygen.

In certain embodiments, the liquid interface polymerization module includes a continuous liquid interface production (CLIP) system such as that described in International Patent Publication No. WO 2014/126837; U.S. Patent Publication Nos. 2018/0064920; 2017/0095972; 2021/0246252 and U.S. Patent Publication Nos. 10,155,882; 10,792,857, the disclosures of which are herein incorporated by reference.

Aspects of the present disclosure further include computer-controlled systems, where the systems further include one or more computers for complete automation or partial automation of the methods described herein. In embodiments, the system includes an input module, a processing module and an output module. The subject systems may include both hardware and software components, where the hardware components may take the form of one or more platforms, e.g., in the form of servers, such that the functional elements, i.e., those elements of the system that carry out specific tasks (such as managing input and output of information, processing information, etc.) of the system may be carried out by the execution of software applications on and across the one or more computer platforms represented of the system.

Systems may include a display and operator input device. Operator input devices may, for example, be a keyboard, mouse, or the like. The processing module includes a processor which has access to a memory having instructions stored thereon for performing the steps of the subject methods. The processing module may include an operating system, a graphical user interface (GUI) controller, a system memory, memory storage devices, and input-output controllers, cache memory, a data backup unit, and many other devices. The processor may be a commercially available processor or it may be one of other processors that are or will become available. The processor executes the operating system and the operating system interfaces with firmware and hardware in a well-known manner, and facilitates the processor in coordinating and executing the functions of various computer programs that may be written in a variety of programming languages, such as Java, Perl, C++, other high level or low level languages, as well as combinations thereof, as is known in the art. The operating system, typically in cooperation with the processor, coordinates and executes functions of the other components of the computer. The operating system also provides scheduling, input-output control, file and data management, memory management, and communication control and related services, all in accordance with known techniques. The processor may be any suitable analog or digital system.

The system memory may be any of a variety of known or future memory storage devices. Examples include any commonly available random access memory (RAM), magnetic medium such as a resident hard disk or tape, an optical medium such as a read and write compact disc, flash memory devices, or other memory storage device. The memory storage device may be any of a variety of known or future devices, including a compact disk drive, a tape drive, a removable hard disk drive, or a diskette drive. Such types of memory storage devices typically read from, and/or write to, a program storage medium (not shown) such as, respectively, a compact disk, magnetic tape, removable hard disk, or floppy diskette. Any of these program storage media, or others now in use or that may later be developed, may be considered a computer program product. As will be appreciated, these program storage media typically store a computer software program and/or data. Computer software programs, also called computer control logic, typically are stored in system memory and/or the program storage device used in conjunction with the memory storage device.

In some embodiments, a computer program product is described having a computer usable medium having control logic (computer software program, including program code) stored therein. The control logic, when executed by the processor the computer, causes the processor to perform functions described herein. In other embodiments, some functions are implemented primarily in hardware using, for example, a hardware state machine. Implementation of the hardware state machine so as to perform the functions described herein will be apparent to those skilled in the relevant arts.

Memory may be any suitable device in which the processor can store and retrieve data, such as magnetic, optical, or solid-state storage devices (including magnetic or optical disks or tape or RAM, or any other suitable device, either fixed or portable). The processor may include a general-purpose digital microprocessor suitably programmed from a computer readable medium carrying necessary program code. Programming can be provided remotely to processor through a communication channel, or previously saved in a computer program product such as memory or some other portable or fixed computer readable storage medium using any of those devices in connection with memory. For example, a magnetic or optical disk may carry the programming, and can be read by a disk writer/reader. Systems of the invention also include programming, e.g., in the form of computer program products, algorithms for use in practicing the methods as described above. Programming according to the present invention can be recorded on computer readable media, e.g., any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; portable flash drive; and hybrids of these categories such as magnetic/optical storage media.

The processor may also have access to a communication channel to communicate with a user at a remote location. By remote location is meant the user is not directly in contact with the system and relays input information to an input manager from an external device, such as a computer connected to a Wide Area Network (“WAN”), telephone network, satellite network, or any other suitable communication channel, including a mobile telephone (i.e., smartphone).

In some embodiments, systems according to the present disclosure may be configured to include a communication interface. In some embodiments, the communication interface includes a receiver and/or transmitter for communicating with a network and/or another device. The communication interface can be configured for wired or wireless communication, including, but not limited to, radio frequency (RF) communication (e.g., Radio-Frequency Identification (RFID), Zigbee communication protocols, WiFi, infrared, wireless Universal Serial Bus (USB), Ultra Wide Band (UWB), Bluetooth® communication protocols, and cellular communication, such as code division multiple access (CDMA) or Global System for Mobile communications (GSM).

In one embodiment, the communication interface is configured to include one or more communication ports, e.g., physical ports or interfaces such as a USB port, an RS-232 port, or any other suitable electrical connection port to allow data communication between the subject systems and other external devices such as a computer terminal (for example, at a physician's office or in hospital environment) that is configured for similar complementary data communication.

In one embodiment, the communication interface is configured for infrared communication, Bluetooth® communication, or any other suitable wireless communication protocol to enable the subject systems to communicate with other devices such as computer terminals and/or networks, communication enabled mobile telephones, personal digital assistants, or any other communication devices which the user may use in conjunction.

In one embodiment, the communication interface is configured to provide a connection for data transfer utilizing Internet Protocol (IP) through a cell phone network, Short Message Service (SMS), wireless connection to a personal computer (PC) on a Local Area Network (LAN) which is connected to the internet, or WiFi connection to the internet at a WiFi hotspot.

In one embodiment, the subject systems are configured to wirelessly communicate with a server device via the communication interface, e.g., using a common standard such as 802.11 or Bluetooth® RF protocol, or an IrDA infrared protocol. The server device may be another portable device, such as a smart phone, Personal Digital Assistant (PDA) or notebook computer; or a larger device such as a desktop computer, appliance, etc. In some embodiments, the server device has a display, such as a liquid crystal display (LCD), as well as an input device, such as buttons, a keyboard, mouse or touch-screen.

In some embodiments, the communication interface is configured to automatically or semi-automatically communicate data stored in the subject systems, e.g., in an optional data storage unit, with a network or server device using one or more of the communication protocols and/or mechanisms described above.

Output controllers may include controllers for any of a variety of known display devices for presenting information to a user, whether a human or a machine, whether local or remote. If one of the display devices provides visual information, this information typically may be logically and/or physically organized as an array of picture elements. A graphical user interface (GUI) controller may include any of a variety of known or future software programs for providing graphical input and output interfaces between the system and a user, and for processing user inputs. The functional elements of the computer may communicate with each other via system bus. Some of these communications may be accomplished in alternative embodiments using network or other types of remote communications. The output manager may also provide information generated by the processing module to a user at a remote location, e.g., over the Internet, phone or satellite network, in accordance with known techniques. The presentation of data by the output manager may be implemented in accordance with a variety of known techniques. As some examples, data may include SQL, HTML or XML documents, email or other files, or data in other forms. The data may include Internet URL addresses so that a user may retrieve additional SQL, HTML, XML, or other documents or data from remote sources. The one or more platforms present in the subject systems may be any type of known computer platform or a type to be developed in the future, although they typically will be of a class of computer commonly referred to as servers. However, they may also be a main-frame computer, a work station, or other computer type. They may be connected via any known or future type of cabling or other communication system including wireless systems, either networked or otherwise. They may be co-located or they may be physically separated. Various operating systems may be employed on any of the computer platforms, possibly depending on the type and/or make of computer platform chosen. Appropriate operating systems include Windows NT®, Windows XP, Windows 7, Windows 8, iOS, Sun Solaris, Linux, OS/400, Compaq Tru64 Unix, SGI IRIX, Siemens Reliant Unix, and others.

### Polymeric Structures Having Resolved Micro-Void Space

Aspects of the present disclosure also include polymeric structures having a resolved micro-void space prepared by the subject methods described herein. In some instances, the polymeric structure includes a plurality of micro-void spaces. In some instances, the micro-void space includes one or more distinct microchannels, such as 2 or more, such as 3 or more, such as 4 or more, such as 5 or more and including 10 or more distinct microchannels. In some instances, one or more of the microchannels includes one or more bifurcations, such as 2 or more bifurcations, such as 3 or more, such as 4 or more, such as 5 or more and including 10 or more bifurcations. In some instances, the microchannels extend through the polymeric structure. In some instances, the microchannels are fluidically interconnected. In some instances, the polymeric structure has a single network of fluidically interconnected microchannel networks. In other instances, the polymeric structure has a plurality of fluidically interconnected microchannel networks. In some embodiments, each microchannel has a diameter of 0.01 μm or more, such as 0.05 μm or more, such as 0.1 μm or more, such as 0.5 μm or more, such as 1 μm or more, such as 2 μm or more, such as 3 μm or more, such as 4 μm or more, such as 5 μm or more, such as 10 μm or more, such as 15 μm or more, such as 20 μm or more, such as 25 μm or more, such as 50 μm or more, such as 75 μm or more and including 100 μm or more. In certain instances, each microchannel has a diameter of from 0.01 μm to 75 μm, such as from 0.05 μm to 50 μm, such as from 0.1 μm to 25 μm, such as from 0.5 μm to 20 μm.

Depending on the size of the polymeric structure and micro-void space, each resolved micro-void space has a volume of 0.001 μL or more, such as 0.005 μL or more, such as 0.01 UL or more, such as 0.05 μL or more, such as 0.1 μL or more, such as 0.2 μL or more, such as 0.3 μL or more, such as 0.4 μL or more, such as 0.5 μL or more, such as 1 μL or more, such as 2 μL or more, such as 3 μL or more, such as 4 μL or more, such as 5 μL or more, such as 6 μL or more, such as 7 μL or more, such as 8 μL or more, such as 9 μL or more, such as 10 μL or more, such as 15 μL or more, such as 20 μL or more and including 25 μL or more. In some instances, the volume of each micro-void space has a volume from 0.01 μL to 2.5 μL, such as from 0.02 μL to 2.4 μL, such as from 0.03 μL to 2.3 μL, such as from 0.04 μL to 2.2 μL, such as rom 0.05 μL to 2.1 μL, such as from 0.06 μL to 2.0 L, such as from 0.07 μL to 1.9 μL, such as from 0.08 μL to 1.8 μL, such as from 0.09 μL to 1.7 L and including where each micro-void space has a volume of from 1 μL to 1.5 L. In some instances, the resolved micro-void space in the polymeric structure has a cumulative volume (i.e., the volume of all of the micro-void space together) of 0.1 μL or more, such as 0.2 μL or more, such as 0.3 μL or more, such as 0.4 μL or more, such as 0.5 μL or more, such as 1 μL or more, such as 2 μL or more, such as 3 μL or more, such as 4 μL or more, such as 5 L or more, such as 6 μL or more, such as 7 μL or more, such as 8 μL or more, such as 9 μL or more, such as 10 UL or more, such as 15 μL or more, such as 20 L or more and including 25 μL or more.

In some instances, polymeric structures of interest include a non-polymerizable composition positioned therein. In some instances, the non-polymerizable composition fills 5% or more of the void volume (i.e., the negative space within the polymeric structure) of the micro-void space, such as 10% or more, such as 15% or more, such as 20% or more, such as 25% or more, such as 50% or more, such as 75% or more, such as 90% or more, such as 95% or more, such as 97% or more, such as 99% or more and including, where the non-polymerizable composition fills the entire volume of the void volume of the micro-void space. In some instances, the non-polymerizable composition is non-reactive with the polymeric structure. In some instances, the non-polymerizable composition positioned within the void volume of the polymeric structure is water, a Newtonian liquid, a shear thinning liquid, a shear thickening liquid, a magnetorheological liquid, an electric field responsive liquid and a gas. In certain embodiments, aspects of the present disclosure include a polymeric structure having a micro-void space where the non-polymerizable composition has been removed.

In embodiments, the polymeric structure is formed from a polymerizable material which may include but is not limited to polycaprolactone, polyglycolic acid, polylactic acid, polylactic-co-glycolic acid, polyethylene glycol, thiol-enes, anhydrides, polyacrylic acid, poly methylmethacrylate, polyvinyl alcohol, polyvinylpyrrolidone, vinyl carbonates, vinyl esters, acrylamides, hyaluronic acid, chitosan, collagen, gelatin, carboxymethylcellulose, and blends or copolymers thereof. In certain embodiments, the polymeric structure is formed from polyethylene glycol dimethacrylate (PEGDMA). In certain embodiments, the polymeric structure is formed from trimethylolpropane triacrylate (TMPTA) monomer. In certain embodiments, the polymerizable material is selected from polycarbonates, polyvinyl chloride (PVC), polyurethanes, polyethers, polyamides, polyimides, or copolymers of these thermoplastics, such as PETG (glycol-modified polyethylene terephthalate), among other polymeric plastic materials. In certain embodiments, the beamsplitter is formed from a polyester, where polyesters of interest may include, but are not limited to, poly(alkylene terephthalates) such as poly(ethylene terephthalate) (PET), bottle-grade PET (a copolymer made based on monoethylene glycol, terephthalic acid, and other comonomers such as isophthalic acid, cyclohexene dimethanol, etc.), poly(butylene terephthalate) (PBT), and poly(hexamethylene terephthalate); poly(alkylene adipates) such as poly(ethylene adipate), poly(1,4-butylene adipate), and poly(hexamethylene adipate); poly(alkylene suberates) such as poly(ethylene suberate); poly(alkylene sebacates) such as poly(ethylene sebacate); poly(ε-caprolactone) and poly(β-propiolactone); poly(alkylene isophthalates) such as poly(ethylene isophthalate); poly(alkylene 2,6-naphthalene-dicarboxylates) such as poly(ethylene 2,6-naphthalene-dicarboxylate); poly(alkylene sulfonyl-4,4′-dibenzoates) such as poly(ethylene sulfonyl-4,4′-dibenzoate); poly(p-phenylene alkylene dicarboxylates) such as poly(p-phenylene ethylene dicarboxylates); poly(trans-1,4-cyclohexanediyl alkylene dicarboxylates) such as poly(trans-1,4-cyclohexanediyl ethylene dicarboxylate); poly(1,4-cyclohexane-dimethylene alkylene dicarboxylates) such as poly(1,4-cyclohexane-dimethylene ethylene dicarboxylate); poly([2.2.2]-bicyclooctane-1,4-dimethylene alkylene dicarboxylates) such as poly([2.2.2]-bicyclooctane-1,4-dimethylene ethylene dicarboxylate); lactic acid polymers and copolymers such as(S)-polylactide, (R,S)-polylactide, poly(tetramethylglycolide), and poly(lactide-co-glycolide); and polycarbonates of bisphenol A, 3,3′-dimethylbisphenol A, 3,3′,5,5′-tetrachlorobisphenol A, 3,3′,5,5′-tetramethylbisphenol A; polyamides such as poly(p-phenylene terephthalamide); polyethylene Terephthalate (e.g., Mylar™ Polyethylene Terephthalate), combinations thereof, and the like. In some embodiments, the polymeric structure is formed from one or more polymerizable materials, such as two or more different polymerizable materials, such as three or more and including four or more different polymerizable materials. In certain instances, one or more of the polymerizable materials includes carbon nanotubes, such as single-walled carbon nanotubes (SWCNTs) or multi-walled carbon nanotubes (MWCNTs).

### Non-Transitory Computer-Readable Storage Medium

Aspects of the present disclosure further include non-transitory computer readable storage mediums having instructions for practicing the subject methods. Computer readable storage mediums may be employed on one or more computers for complete automation or partial automation of a system for practicing methods described herein. In certain embodiments, instructions in accordance with the method described herein can be coded onto a computer-readable medium in the form of “programming”, where the term “computer readable medium” as used herein refers to any non-transitory storage medium that participates in providing instructions and data to a computer for execution and processing. Examples of suitable non-transitory storage media include a floppy disk, hard disk, optical disk, magneto-optical disk, CD-ROM, CD-R, magnetic tape, non-volatile memory card, ROM, DVD-ROM, Blue-ray disk, solid state disk, and network attached storage (NAS), whether or not such devices are internal or external to the computer. A file containing information can be “stored” on computer readable medium, where “storing” means recording information such that it is accessible and retrievable at a later date by a computer. The computer-implemented method described herein can be executed using programming that can be written in one or more of any number of computer programming languages. Such languages include, for example, Python, Java, Java Script, C, C#, C++, Go, R, Swift, PHP, as well as any many others.

In some instances, the non-transitory computer readable storage medium has instructions stored thereon that include: algorithm for irradiating a polymerizable composition positioned between a build elevator and a build surface to generate a polymerizable composition having a polymerized region of the polymerizable composition with a micro-void space in contact with the build elevator and a non-polymerized region of the polymerizable composition in contact with the build surface, algorithm for displacing the build elevator away from the build surface, algorithm for contacting the generated micro-void space with a non-reactive composition and algorithm for repeating one or more steps in a manner sufficient to generate a polymeric structure having a resolved micro-void space. In some instances, the non-transitory computer readable storage medium has algorithm for injecting the polymerizable composition through the conduit with a syringe pump. In some instances, the non-transitory computer readable storage medium has algorithm for generating a polymeric structure having a micro-void space that includes a non-polymerizable composition positioned therein.

In some embodiments, the non-transitory computer readable storage medium has algorithm for contacting the generated micro-void space with an amount of the polymerizable composition in a manner sufficient to displace polymerized material in the micro-void space. In some instances, the non-transitory computer readable storage medium has algorithm for continuously conveying the polymerizable composition through the generated micro-void space to displace polymerized material in the micro-void space. In some instances, the non-transitory computer readable storage medium has algorithm for injecting polymerizable composition the generated micro-void space to displace polymerized material in the micro-void space. In some instances, the non-transitory computer readable storage medium has algorithm for injecting the polymerizable composition into the generated micro-void space with a syringe. In some instances, the non-transitory computer readable storage medium has algorithm for continuously conveying the polymerizable composition through the generated micro-void space into the space between the build elevator and the build surface of the liquid interface production module.

In some instances, the non-transitory computer readable storage medium has algorithm for contacting a non-polymerizable composition with the generated micro-void space of the polymeric structure. In some instances, the non-transitory computer readable storage medium has algorithm for filling at least a part of the void volume of the micro-void space, such as 5% or more, such as 10% or more, such as 25% or more, such as 50% or more and including 75% or more of the void volume of the micro-void space with the non-polymerizable composition, such as for filling the entire void volume of the micro-void space with the non-polymerizable composition. In some embodiments, the non-transitory computer readable storage medium has algorithm for generating a polymeric structure having a plurality of micro-void spaces. In certain instances, the non-transitory computer readable storage medium has algorithm for irradiating the polymerizable composition for a duration sufficient to bond the first polymerized region of the polymerizable composition to the build elevator. In some instances, the non-transitory computer readable storage medium has algorithm for displacing the build elevator in predetermined increments of from 0.5 μm to 1.0 μm. In some embodiments, the non-transitory computer readable storage medium has algorithm for adding polymerizable composition to the build surface after each displacement of the build elevator away from the build surface. In some instances, the non-transitory computer readable storage medium has algorithm for adding non-polymerizable composition to the generated micro-void space after each displacement of the build elevator away from the build surface. In some instances, the non-transitory computer readable storage medium has algorithm for continuously polymerizing the polymerizable composition while displacing the build elevator away from the build surface. In some embodiments, the non-transitory computer readable storage medium has algorithm for continuously adding non-polymerizable composition to the generated micro-void space while displacing the build elevator away from the build surface. In certain embodiments, the non-transitory computer readable storage medium has algorithm for removing the non-polymerizable composition from the generated micro-void space of the polymeric structure.

The non-transitory computer readable storage medium may be employed on one or more computer systems having a display and operator input device. Operator input devices may, for example, be a keyboard, mouse, or the like. The processing module includes a processor which has access to a memory having instructions stored thereon for performing the steps of the subject methods. The processing module may include an operating system, a graphical user interface (GUI) controller, a system memory, memory storage devices, and input-output controllers, cache memory, a data backup unit, and many other devices. The processor may be a commercially available processor or it may be one of other processors that are or will become available. The processor executes the operating system and the operating system interfaces with firmware and hardware in a well-known manner, and facilitates the processor in coordinating and executing the functions of various computer programs that may be written in a variety of programming languages, such as those mentioned above, other high level or low level languages, as well as combinations thereof, as is known in the art. The operating system, typically in cooperation with the processor, coordinates and executes functions of the other components of the computer. The operating system also provides scheduling, input-output control, file and data management, memory management, and communication control and related services, all in accordance with known techniques.

### Kits

Kits for use in practicing certain methods described herein are also provided. In certain embodiments, the kits include one or more polymeric structures as described above. In some instances, kits include a polymerizable composition for preparing the polymeric structures. In certain embodiments, kits include a non-reactive composition, such as a non-polymerizable composition for contacting with the generated micro-void space. For example, kits may further include one or more of an amount of water, a Newtonian liquid, a shear thinning liquid, a shear thickening liquid, a magnetorheological liquid, an electric field responsive liquid and a gas.

In certain embodiments, the kits will further include instructions for practicing the subject methods or means for obtaining the same (e.g., a website URL directing the user to a webpage which provides the instructions), where these instructions may be printed on a substrate, where substrate may be one or more of: a package insert, the packaging, reagent containers and the like. Yet another form of these instructions is a computer readable medium, e.g., diskette, compact disk (CD), portable flash drive, USB storage, DVD, Blu-ray disk, etc.), and the like, on which the information has been recorded. Yet another form of these instructions that may be present is a website address which may be used via the internet to access the information at a removed site.

Notwithstanding the appended claims, the disclosure is also defined by the following clauses:
1. A method of making a polymeric structure comprising a micro-void space, the method comprising:
   a) irradiating a polymerizable composition positioned between a build elevator and a build surface of a liquid interface production module to generate a polymerizable composition comprising a polymerized region of the polymerizable composition comprising a micro-void space in contact with the build elevator and a non-polymerized region of the polymerizable composition in contact with the build surface;
   b) displacing the build elevator away from the build surface;
   c) contacting the generated micro-void space with a non-reactive composition; and
   d) repeating steps a)-c) in a manner sufficient to generate a polymeric structure having a resolved micro-void space.
2. The method according to 1, wherein the generated micro-void space comprises one or more microchannels in the polymeric structure.
3. The method according to 2, wherein one or more of the microchannels extends through the polymeric structure.
4. The method according to any one of 1-3, wherein the method comprises contacting the generated micro-void space with an amount of the polymerizable composition in a manner sufficient to displace polymerized material in the micro-void space.
5. The method according to 4, wherein the amount of polymerizable composition is continuously conveyed through the generated micro-void space to displace polymerized material in the micro-void space.
6. The method according to any one of 2-5, wherein the polymerizable composition is injected through the generated micro-void space.
7. The method according to any one of 2-6, wherein the polymerizable composition is conveyed through the generated micro-void space into the space between the build elevator and the build surface of the liquid interface production module.
8. The method according to any one of 6-7, wherein the polymerizable composition is injected through the generated micro-void space with a syringe.
9. The method according to 8, wherein the syringe is operationally coupled to a syringe pump.
10. The method according to any one of 1-3, wherein the method comprises contacting the generated micro-void space with a non-polymerizable composition.
11. The method according to 10, wherein the non-polymerizable composition is contacted with the generated micro-void space while generating the polymeric structure.
12. The method according to 11, wherein the non-polymerizable composition is continuously contacted with the micro-void space while generating the polymeric structure.
13. The method according to any one of 10-12, wherein the method comprises filling at least a portion of the void volume of the micro-void space with the non-polymerizable composition.
14. The method according to 13, wherein the method comprises filling 5% or more of the void volume of the micro-void space with the non-polymerizable composition.
15. The method according to any one of 10-14, wherein the non-polymerizable composition is non-reactive with the polymerizable composition of the polymeric structure.
16. The method according to any one of 10-15, wherein the non-polymerizable composition is selected from the group consisting of water, a Newtonian liquid, a shear thinning liquid, a shear thickening liquid, a magnetorheological liquid, an electric field responsive liquid and a gas.
17. The method according to any one of 10-16, wherein the method further comprises removing the non-polymerizable composition from the generated micro-void space of the polymeric structure.
18. The method according to any one of 1-17, wherein the polymeric structure comprises a plurality of micro-void spaces.
19. The method according to any one of 1-18, wherein the polymerizable composition is in contact with the build elevator and the build surface.
20. The method according to 19, wherein the method comprises irradiating the polymerizable composition for a duration sufficient to bond the first polymerized region of the polymerizable composition to the build elevator.
21. The method according to any one of 1-20, wherein the build elevator is displaced in predetermined increments of from 0.5 μm to 1.0 μm.
22. The method according to any one of 1-21, wherein the method further comprises adding polymerizable composition to the build surface after each displacement of the build elevator away from the build surface.
23. The method according to 22, wherein the method further comprises contacting the generated micro-void space with the non-reactive composition after each displacement of the build elevator away from the build surface.
24. The method according to any one of 1-23, wherein the polymerizable composition is continuously polymerized while displacing the build elevator away from the build surface.
25. The method according to 24, wherein the non-reactive composition is continuously contacted with the generated micro-void space while displacing the build elevator away from the build surface.
26. The method according to any one of 1-25, wherein the polymerizable composition comprises a polymerizable material selected from the group consisting of polycaprolactone, polyglycolic acid, polylactic acid, polylactic-co-glycolic acid, polyethylene glycol, polyethylene glycol dimethacrylate (PEGDMA), thiol-enes, anhydrides, polyacrylic acid, poly methylmethacrylate, trimethylolpropane triacrylate (TMPTA) monomer, polyvinyl alcohol, polyvinylpyrrolidone, vinyl carbonates, vinyl esters, acrylamides, hyaluronic acid, chitosan, collagen, gelatin, carboxymethylcellulose, and blends or copolymers thereof.
27. The method according to 26, wherein the polymerizable composition comprises polyethylene glycol dimethacrylate (PEGDMA).
28. The method according to any one of 26-27, wherein the polymerizable materials comprise carbon nanotubes.
29. The method according to 28, wherein the polymerizable materials comprise one or more of single-walled carbon nanotubes (SWCNTs) or multi-walled carbon nanotubes (MWCNTs).
30. A system for making a polymeric structure comprising a micro-void space, the system comprising:
   a light source; and
   a light interface polymerization module comprising a build elevator and a build surface configured for generating a polymeric structure comprising a resolved micro-void space therein from a polymerizable composition positioned therebetween.
31. The system according to 30, wherein the light interface polymerization module is configured for generating a polymeric structure having one or more microchannels in the polymeric structure.
32. The system according to 31, wherein one or more of the microchannels extends through the polymeric structure.
33. The system according to any one of 30-32, wherein the system further comprises a processor comprising memory operably coupled to the processor wherein the memory comprises instructions stored thereon, which when executed by the processor, cause the processor to:
   a) irradiate a polymerizable composition positioned between a build elevator and a build surface to generate a polymerizable composition comprising a polymerized region of the polymerizable composition comprising a micro-void space in contact with the build elevator and a non-polymerized region of the polymerizable composition in contact with the build surface;
   b) displace the build elevator away from the build surface;
   c) contact the generated micro-void space with a non-reactive composition; and
   d) repeat steps a)-c) in a manner sufficient to generate a polymeric structure having a resolved micro-void space.
34. The system according to 33, wherein the memory comprises instructions for contacting the generated micro-void space with an amount of the polymerizable composition in a manner sufficient to displace polymerized material in the micro-void space.
35. The system according to 34, wherein the memory comprises instructions for continuously conveying the polymerizable composition through the generated micro-void space to displace polymerized material in the micro-void space.
36. The system according to any one of 33-35, wherein the polymerizable composition is injected through the generated micro-void space.
37. The system according to any one of 33-36, wherein the memory comprises instructions for continuously conveying the polymerizable composition through the generated micro-void space into the space between the build elevator and the build surface of the liquid interface production module.
38. The system according to any one of 30-37, wherein the system further comprises a syringe pump.
39. The system according to any one of 30-33, wherein the memory comprises instructions for contacting the generated micro-void space with a non-polymerizable composition.
40. The system according to 39, wherein the system further comprises a source of the non-polymerizable composition that is operably coupled to the light interface polymerization module such that the non-polymerizable composition is continuously contacted with the micro-void space while generating the polymeric structure.
41. The system according to any one of 39-40, wherein the memory comprises instructions for filling at least a part of the void volume of the micro-void space with the non-polymerizable composition.
42. The system according to 41, wherein the memory comprises instructions for filling 5% or more of the void volume of the micro-void space with the non-polymerizable composition.
43. The system according to any one of 39-42, wherein the non-polymerizable composition is non-reactive with the polymerizable composition of the polymeric structure.
44. The system according to any one of 39-43, wherein the non-polymerizable composition is selected from the group consisting of water, a Newtonian liquid, a shear thinning liquid, a shear thickening liquid, a magnetorheological liquid, an electric field responsive liquid and a gas.
45. The system according to any one of 33-44, wherein the memory comprises instructions for generating a polymeric structure comprising a plurality of micro-void spaces.
46. The system according to any one of 33-45, wherein the memory comprises instructions for irradiating the polymerizable composition for a duration sufficient to bond the first polymerized region of the polymerizable composition to the build elevator.
47. The system according to any one of 33-46, wherein the memory comprises instructions for displacing the build elevator in predetermined increments of from 0.5 μm to 1.0 μm.
48. The system according to any one of 33-47, wherein the memory comprises instructions for contacting the generated micro-void space with the fluidic composition after each displacement of the build elevator away from the build surface.
49. The system according to any one of 33-48, wherein the memory comprises instructions for adding polymerizable composition to the build surface after each displacement of the build elevator away from the build surface.
50. The system according to 49, wherein the memory comprises instructions for adding non-polymerizable composition to the generated micro-void space after each displacement of the build elevator away from the build surface.
51. The system according to any one of 33-50, wherein the memory comprises instructions for continuously polymerizing the polymerizable composition while displacing the build elevator away from the build surface.
52. The system according to 51, wherein the memory comprises instructions for continuously contacting the non-reactive composition with the generated micro-void space while displacing the build elevator away from the build surface.
53. The system according to any one of 33-52, wherein the memory comprises instructions for removing the non-polymerizable composition from the generated micro-void space of the polymeric structure.
54. The system according to any one of 30-53, wherein the system comprises a micro-digital light projection system comprising:
   a light beam generator component; and
   a light projection monitoring component.
55. The system according to 54, wherein the light beam generator component comprises:
   a light source;
   a tube lens; and
   one or more projection lenses.
56. The system according to any one of 54-55, wherein the light beam generator component comprises two projection lenses.
57. The system according to 56, wherein the projection lenses are magnification lenses.
58. The system according to 57, wherein the projection lenses provide for 2-fold to 10-fold magnification.
59. The system according to any one of 54-58, wherein the light projection monitoring component comprises a photodetector.
60. The system according to 59, wherein the photodetector comprises a charge-coupled device (CCD).
61. The system according to any one of 30-60, wherein the polymerizable composition comprises a polymerizable material selected from the group consisting of polycaprolactone, polyglycolic acid, polylactic acid, polylactic-co-glycolic acid, polyethylene glycol, polyethylene glycol dimethacrylate (PEGDMA), thiol-enes, anhydrides, polyacrylic acid, poly methylmethacrylate, trimethylolpropane triacrylate (TMPTA) monomer, polyvinyl alcohol, polyvinylpyrrolidone, vinyl carbonates, vinyl esters, acrylamides, hyaluronic acid, chitosan, collagen, gelatin, carboxymethylcellulose, and blends or copolymers thereof.
62. The system according to 61, wherein the polymerizable composition comprises polyethylene glycol dimethacrylate (PEGDMA).
63. The system according to any one of 61-62, wherein the polymerizable materials comprises carbon nanotubes.
64. The system according to 63, wherein the polymerizable materials comprises one or more of single-walled carbon nanotubes (SWCNTs) or multi-walled carbon nanotubes (MWCNTs).
65. A polymeric structure comprising a resolved micro-void space comprising a non-polymerizable composition positioned therein.
66. The polymeric structure according to 65, wherein the non-polymerizable composition fills at least a portion of the void volume of the micro-void space.
67. The polymeric structure according to 66, wherein the non-polymerizable composition comprises fills 5% or more of the void volume of the micro-void space.
68. The polymeric structure according to any one of 65-67, wherein the non-polymerizable composition is non-reactive with the polymeric structure.
69. The polymeric structure according to any one of 65-68, wherein the micro-void space comprises a microchannel within the polymeric structure.
70. The polymeric structure according to 69, wherein the microchannel extends through the polymeric structure.
71. The polymeric structure according to any one of 65-70, wherein the polymeric structure comprises a plurality of micro-void spaces.
72. The polymeric structure according to any one of 65-71, wherein the non-polymerizable composition is selected from the group consisting of a Newtonian liquid, a shear thinning liquid, a shear thickening liquid, a magnetorheological liquid, an electric field responsive liquid and a gas.
73. The polymeric structure according to any one of 65-72, wherein polymeric structure is formed from a polymerizable material selected from the group consisting of polycaprolactone, polyglycolic acid, polylactic acid, polylactic-co-glycolic acid, polyethylene glycol, polyethylene glycol dimethacrylate (PEGDMA), thiol-enes, anhydrides, polyacrylic acid, poly methylmethacrylate, trimethylolpropane triacrylate (TMPTA) monomer, polyvinyl alcohol, polyvinylpyrrolidone, vinyl carbonates, vinyl esters, acrylamides, hyaluronic acid, chitosan, collagen, gelatin, carboxymethylcellulose, and blends or copolymers thereof.
74. The polymeric structure according to 73, wherein the polymeric structure is formed from polyethylene glycol dimethacrylate (PEGDMA).
75. The polymeric structure according to any one of 73-74, wherein the polymerizable materials comprises carbon nanotubes.
76. The polymeric structure according to 75, wherein the polymerizable materials comprises one or more of single-walled carbon nanotubes (SWCNTs) or multi-walled carbon nanotubes (MWCNTs).
77. A polymeric structure comprising a resolved micro-void space.
78. The polymeric structure according to 77, wherein the micro-void space comprises a microchannel within the polymeric structure.
79. The polymeric structure according to 78, wherein the microchannel extends through the polymeric structure.
80. The polymeric structure according to any one of 77-79, wherein the polymeric structure comprises a plurality of micro-void spaces.
81. The polymeric structure according to any one of 77-80, wherein polymeric structure is formed from a polymerizable material selected from the group consisting of polycaprolactone, polyglycolic acid, polylactic acid, polylactic-co-glycolic acid, polyethylene glycol, polyethylene glycol dimethacrylate (PEGDMA), thiol-enes, anhydrides, polyacrylic acid, poly methylmethacrylate, trimethylolpropane triacrylate (TMPTA) monomer, polyvinyl alcohol, polyvinylpyrrolidone, vinyl carbonates, vinyl esters, acrylamides, hyaluronic acid, chitosan, collagen, gelatin, carboxymethylcellulose, and blends or copolymers thereof.
82. The polymeric structure according to 81, wherein the polymeric structure is formed from polyethylene glycol dimethacrylate (PEGDMA).
83. The polymeric structure according to any one of 81-82, wherein the polymerizable materials comprises carbon nanotubes.
84. The polymeric structure according to 83, wherein the polymerizable materials comprises one or more of single-walled carbon nanotubes (SWCNTs) or multi-walled carbon nanotubes (MWCNTs).

### EXPERIMENTAL

The following examples are offered by way of illustration and not by way of limitation. Specifically, the following examples are of specific embodiments for carrying out the present disclosure. The examples are for illustrative purposes only and are not intended to limit the scope of the present disclosure in any way. Efforts have been made to ensure accuracy with respect to numbers used (e.g., amounts, temperatures, etc.), but some experimental error and deviation should, of course, be allowed for.

### High Resolution Stereolithography-Negative Spaces Enabled by Control of Fluid Mechanics

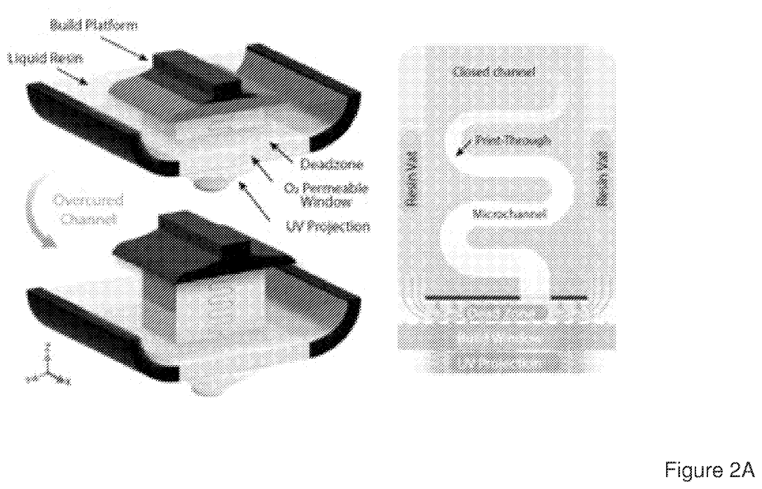

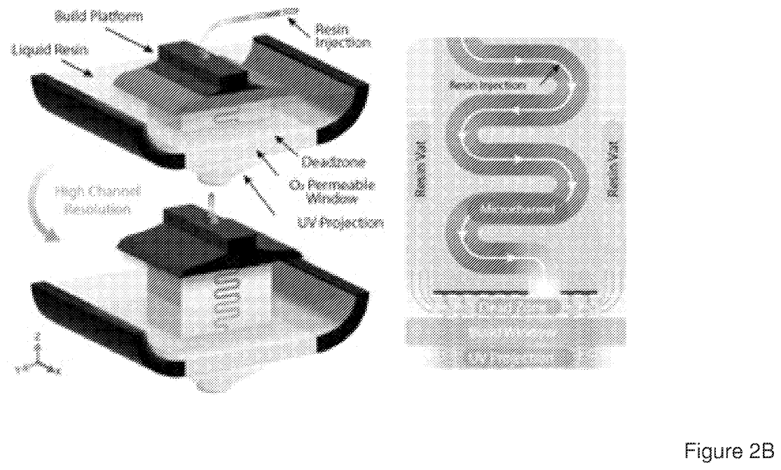

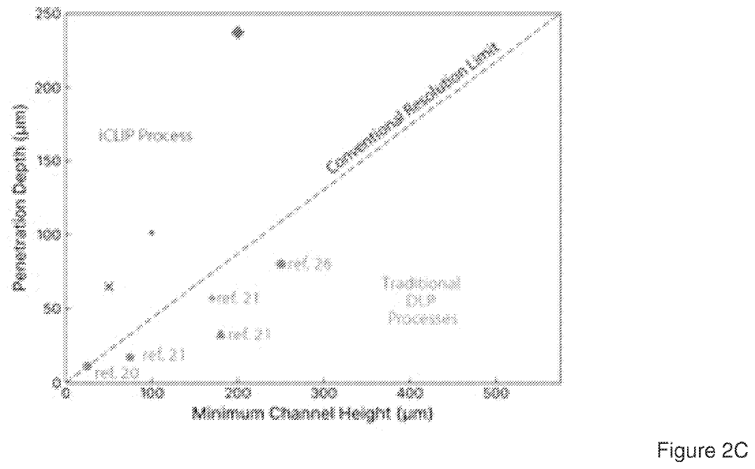

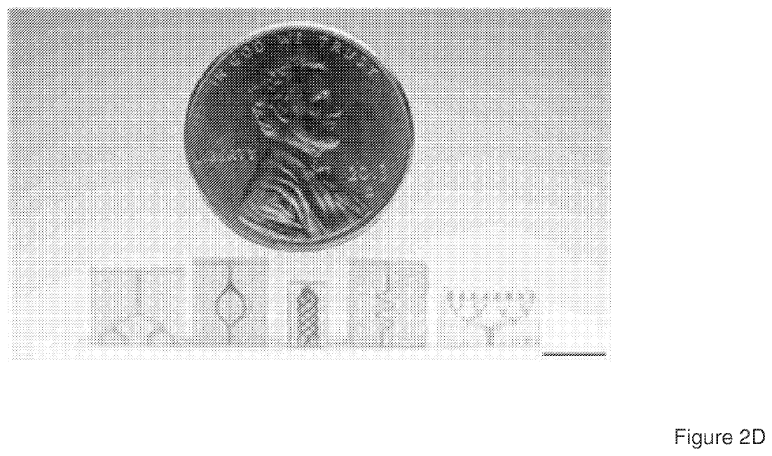

In this example, a method for making polymeric structures having micro-void space (e.g., high-resolution injection CLIP (iCLIP)) is used to achieve micrometer X, Y, and Z resolution using synergistic control of high-resolution optical control and fluid mechanics. Traditional digital light projection (e.g., CLIP) uses projection of ultraviolet (UV) light to cure layers of photopolymerizable resin layer-by-layer. These processes use resin renewal at the build surface through the creation of a continuous liquid interface—the dead zone—resin is drawn into the gap through suction forces created as the curing part is gradually pulled away from the window (FIG. 2A). The dead zone is created and maintained by a constant supply of oxygen—a polymerization inhibitor—that is fed through the highly oxygen-permeable window at the bottom of the resin reservoir. Light delivery is controlled by high-resolution optics that can precisely direct the UV light path to cure a single layer of resin in the X-Y plane with high resolution. As shown in FIG. 2A, light will exponentially decay beyond the intended layer, causing decreased part resolution due to overcuring or print-through in negative spaces (FIG. 2A). Specifically, the penetration depth of a resin represents the characteristic length at which exponential decay occurs within a resin. Print-through has limited the ability of stereolithographic processes to resolve negative relative to the characteristic penetration depth.

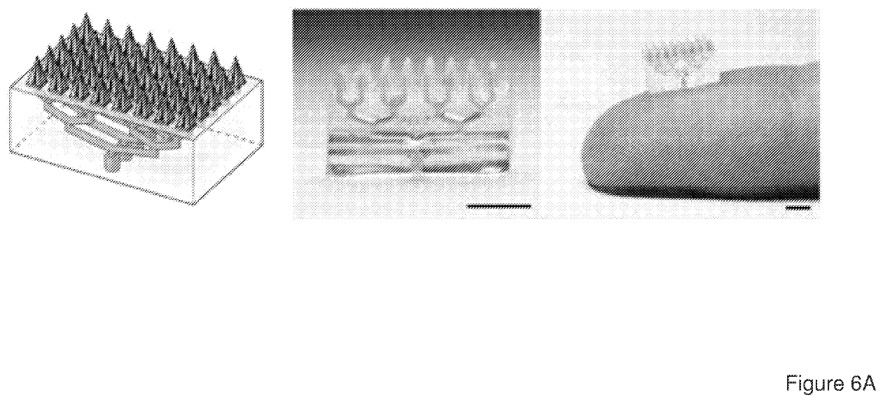

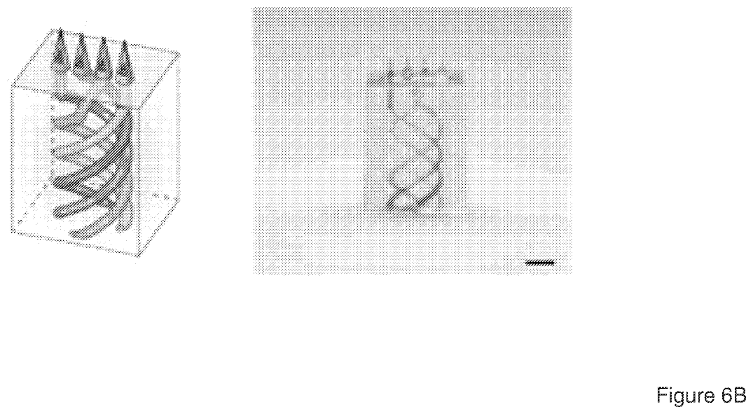

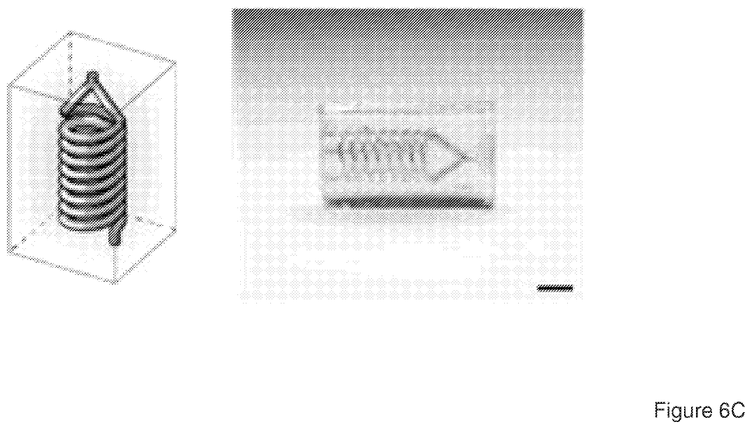

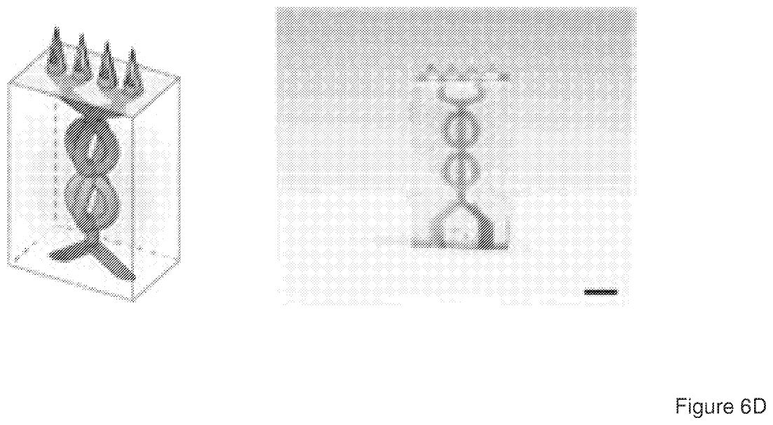

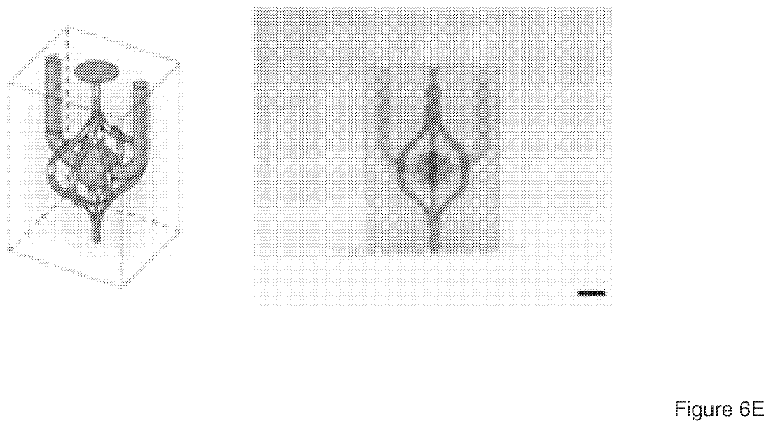

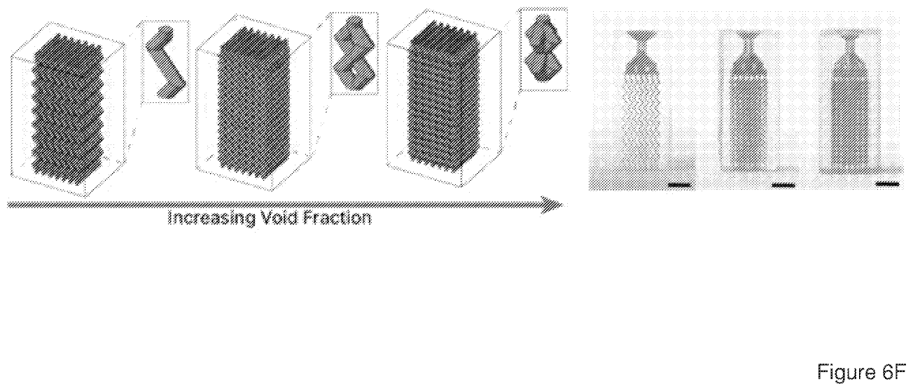

In methods of the present disclosure according to certain embodiments, a stream of fresh polymerizable resin is continuously fed through the build platform to displace trapped resin to preserve designed negative spaces and eliminate print-through (FIG. 2B). Therefore, the methods described herein enable the fabrication of channels with significantly smaller heights/diameters than previously attainable, achieving channel resolutions that match or exceed a resin's penetration depth (FIG. 2C). Fabrication of high-resolution negative spaces using a greater variety of materials enables 3D-printing of high-resolution microsystem devices such as vascular beds and microfluidic-backed microneedles (examples depicted in FIG. 2D and FIGS. 6A-6F).

### Modeling Print-Through

In all DLP-based processes, including CLIP, XY resolution is restricted by the projected pixel size while Z resolution is influenced by the penetration depth of a resin. A larger penetration depth leads to the accumulation of more UV light within the resin, which can unintentionally result in print-through. Using Beer-Lambert's Law, E_{N}, the cumulative UV exposure energy per area after N exposures during part fabrication was determined. The equation for E_{N} is as follows:

$${E}_{N} = {\sum }_{n = 0}^{N} {I}_{0} {te}^{- sn / {D}_{p}} \qquad Equation 1$$

where n is the number of layers from the dead zone, I₀ is the intensity of the UV irradiation at the dead zone, tis the UV exposure time, s is the layer slice thickness of each exposure, and Dₚ is the penetration depth determined by the resin's material properties at the UV wavelength 385 nm.

E_{N} depends on the resin Dₚ, which governs the depth to which UV energy can penetrate and accumulate in negative spaces. Systems of the present disclosure according to certain embodiments displace trapped resin with fresh resin, maintaining a constant turnover that minimizes final E_{N} within microchannels, eliminating the need to reduce a resin's Dₚ for better Z-axis resolution.

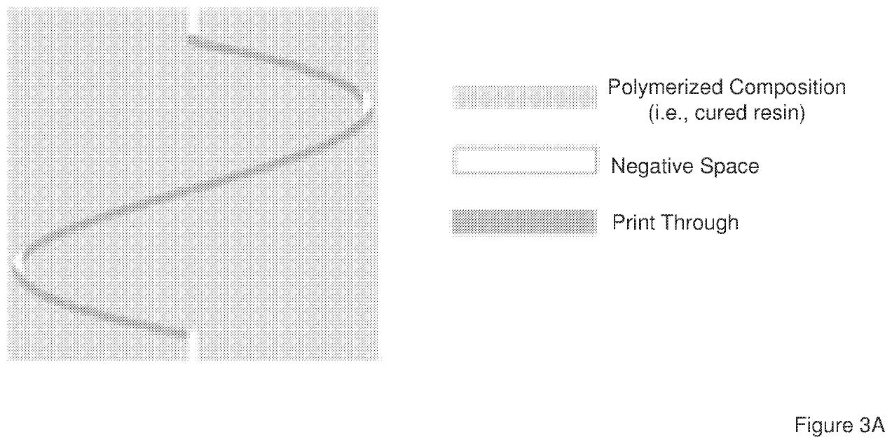

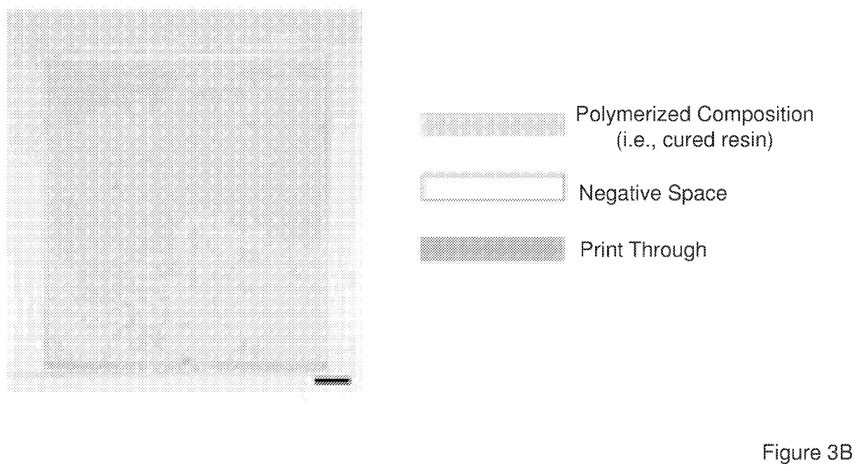

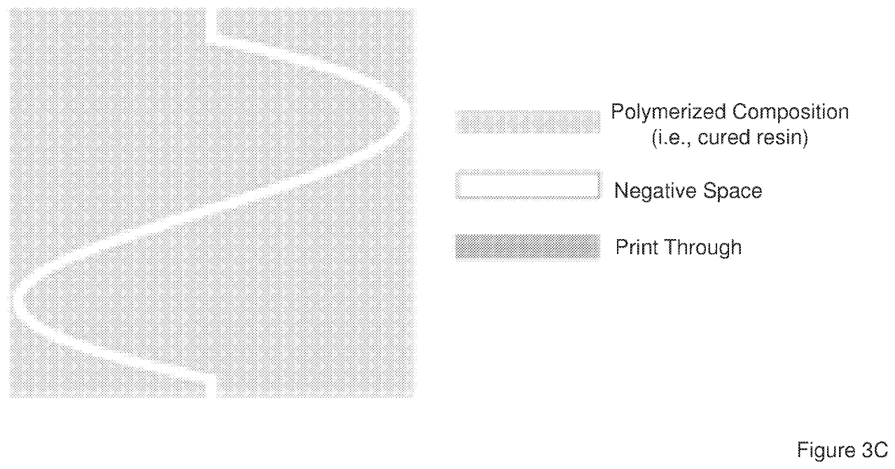

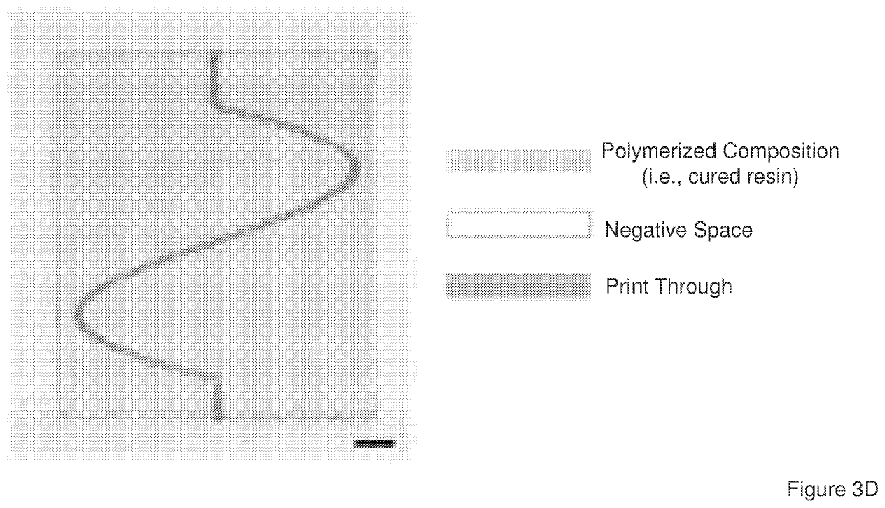

Equation 1 can be used to predict UV dose accumulation within the entire 3D print microstructure. When the critical energy of trapped resin is exceeded, print-through occurs (indicated by blue shading in FIG. 3A). FIG. 3 illustrates dose accumulation in a serpentine microchannel when generating polymeric structures with (iCLIP) and without (CLIP) injecting polymerizable composition through the microchannel during fabrication. Under CLIP conditions, without resin turnover, the accumulation model predicts print-through obstructing the microfluidic channel (FIG. 3A). The model is supported by the resulting CLIP print (FIG. 3B). Conversely, the continuous flow of fresh resin through the microchannel displaces trapped resin before it reaches the critical threshold, preserving the serpentine microchannel and mitigating the print-through effect (FIG. 3C). This is supported by the resulting iCLIP print (FIG. 3D).

### Microchannel Preservation

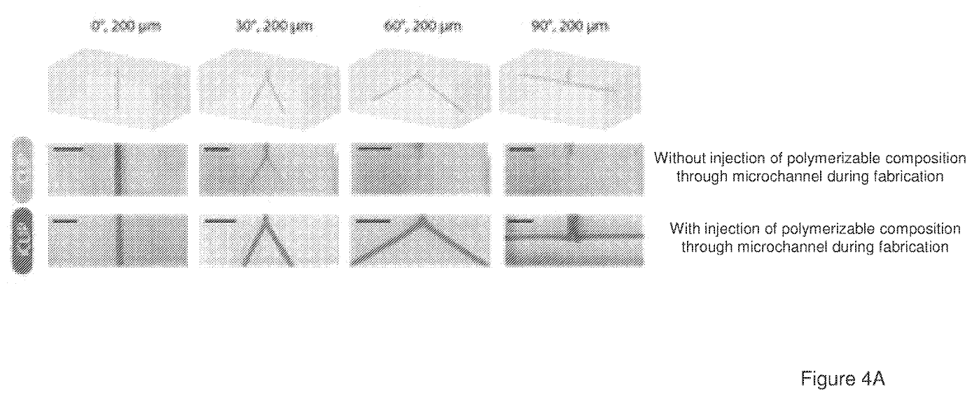

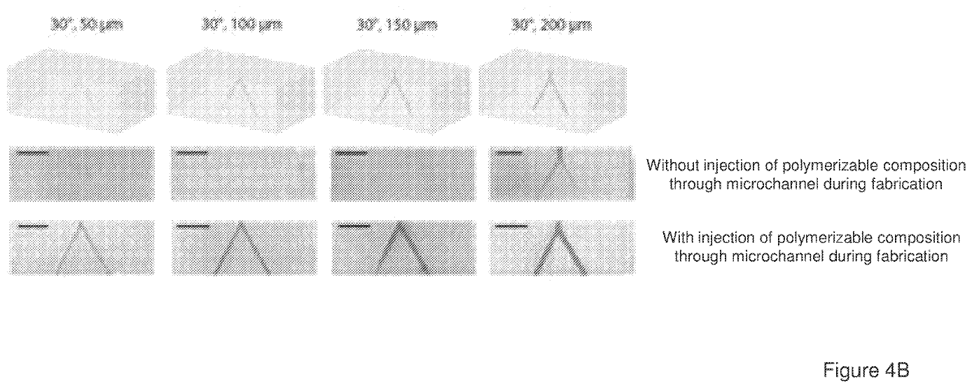

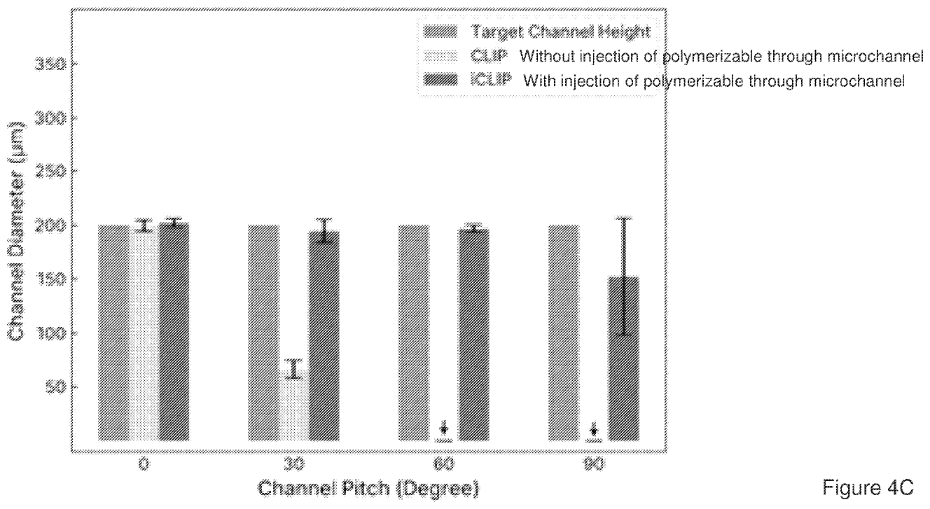

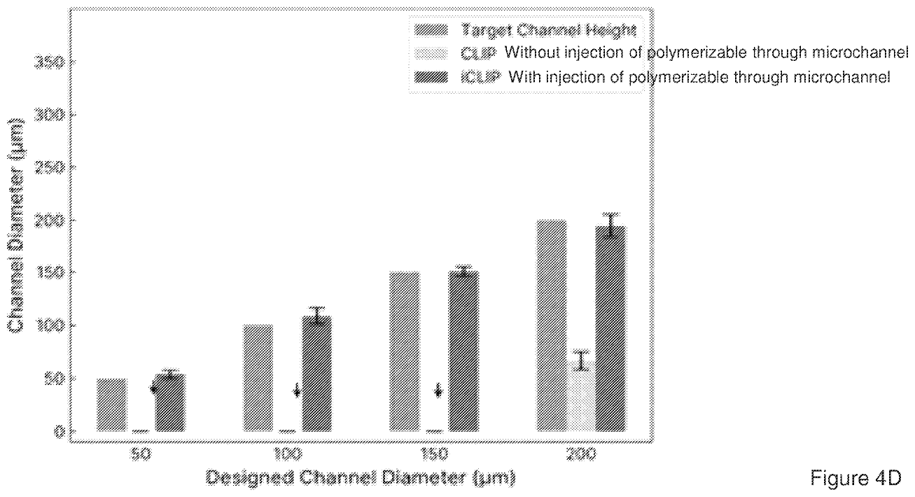

The ability of generating polymeric structures with injection of polymerizable composition through the microchannel during fabrication (iCLIP) to preserve the resolution of negative spaces in a variety of geometric configurations and channel resolutions was investigated. First, to evaluate the performance of iCLIP in resolving diverse microfluidic geometries, microchannels with a diameter of 200 μm were designed and varying pitch angles ranging from 0° to 90° (FIG. 4A). The 0° pitch channel, serving as the control, exhibits the least susceptibility to print-through since it is never exposed to UV light beneath the fabricated channel. In contrast, the 90° pitch channel poses the highest risk of channel obstruction as the vertical z-axis channel height decreases. Optical micrograph images of the cross-sectional profiles of the printed microchannels demonstrate that injection of fresh resin through the microchannel during fabrication (iCLIP) consistently achieves accurate resolution of channels, regardless of their pitch (FIG. 4A and FIG. 4C). The 90° pitch is resolved fully near the injection port while the channel further from the injection source is smaller in diameter. This is postulated to be due to insufficient resin flow before the channel becomes obstructed. To further evaluate the ability of iCLIP to preserve high-resolution negative spaces, a bifurcating microfluidic network with a 30° pitch were designed and printed, varying the channel diameter from 50 μm to 200 μm. Optical micrograph images of the cross-sectional profiles of the printed microchannels confirm accurate microchannel resolution achieved by iCLIP (FIG. 4B and FIG. 4D).

### Process Characterization

To further evaluate generating polymeric structures with injection of polymerizable composition through the microchannel during fabrication (iCLIP), the impact of injection rate of fresh polymerizable resin on channel resolution was determined. A dimensionless turnover number (Tu) represents the ratio of injection rate to fabrication rate of negative space (the rate of microchannel volume being printed). For a given set of print parameters, Tu quantifies the number of print layers cleared by fresh resin before subsequent UV light exposure. For instance, when the injection rate is zero, simulating a traditional CLIP print, Tu=0. When the injection rate matches the fabrication rate, the Tu=1, and when the injection rate exceeds the fabrication rate, the Tu>1. The dimensionless channel diameter is defined as d/D, where d is the resulting channel diameter measured by optical microscopy after printing and D is the designed channel diameter.

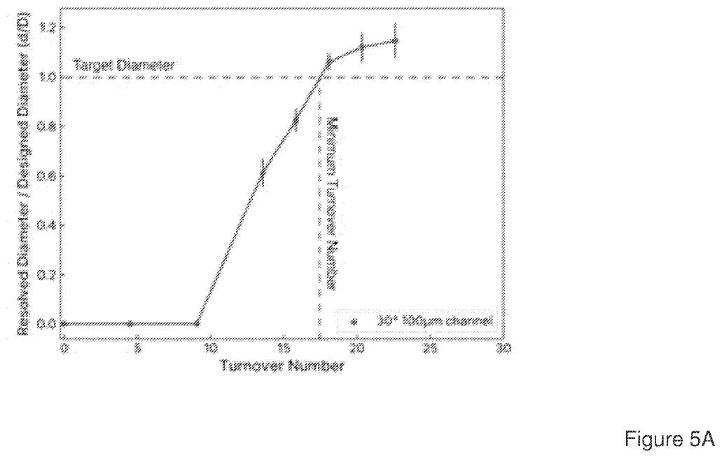

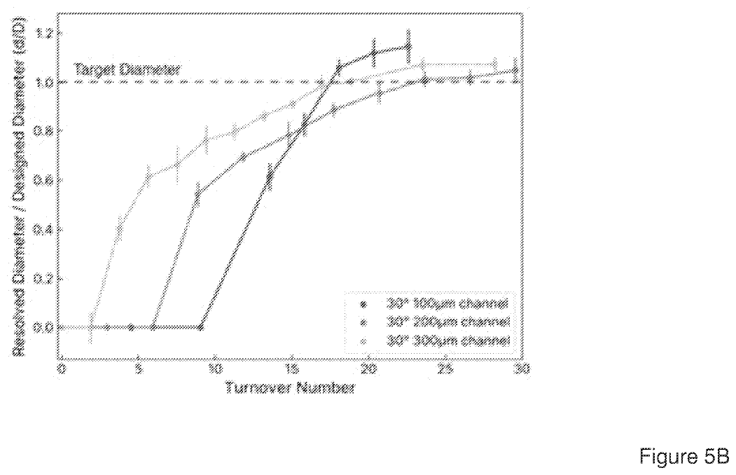

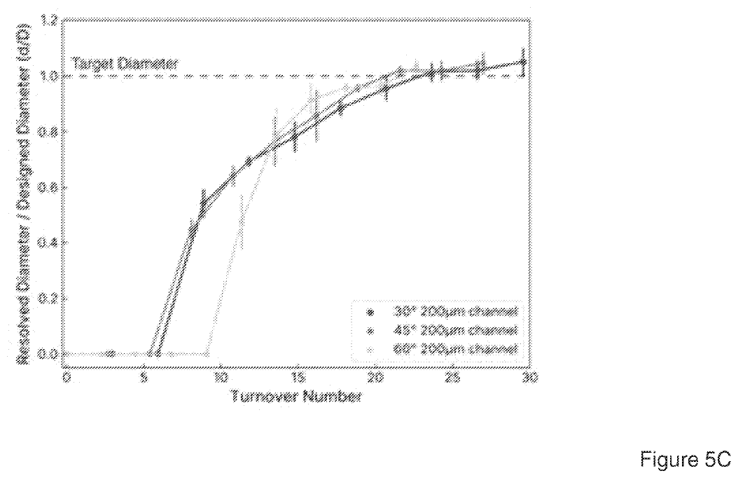

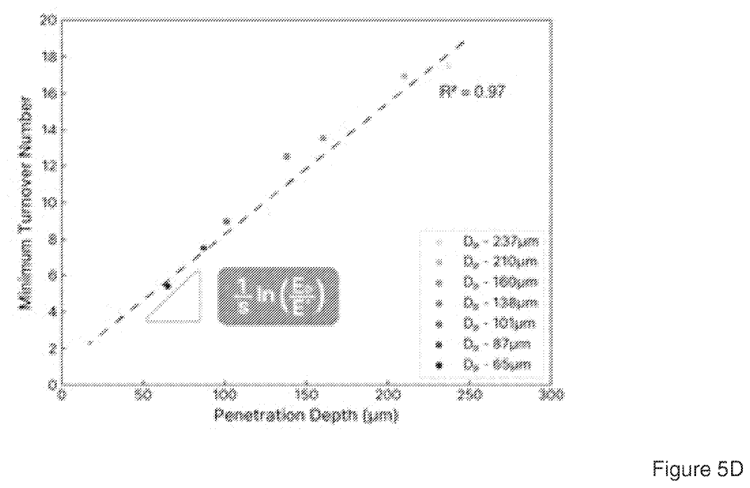

FIG. 5 assesses microchannel resolution in relation to resin turnover when generating polymeric structures having microchannels according to certain embodiments. Under conditions where polymerizable composition (i.e., fresh resin) is not injected (CLIP) through the forming microchannels (Tu=0) led to unresolved channels due to print-through. As Tu increased, d/D approached 1, indicating the minimum Tu required to resolve a given microfluidic structure. Notably, in this case, for a resin with a Dₚ of 237 μm, achieving a Tu greater than 17.5 proved essential for precise microchannel resolution (FIG. 5A). To further explore the impact of Tu, the impact on channel design and geometry on minimum Tu was determined. First, the minimum Tu needed to resolve microfluidic channels with diameters ranging from 100 μm to 300 μm at a 30° pitch was determined (FIG. 5B). Subsequently, the minimum Tu necessary for resolving microchannels with a fixed diameter of 200 μm while varying pitch angles, including 30°, 45°, and 60° was determined (FIG. 5C). Across variations in channel diameter and pitch, the minimum Tu for a resin with a Dₚ of 237 μm exhibited minimal variation, consistently remaining within 15 percent or less of one another.

The impact of Dₚ on the minimum Tu was explored. FIG. 5D illustrates how the Dₚ of various resins affects the required Tu to achieve accurate negative features. To conduct this study, the impact of varying Tu influenced the ability to resolve a 100 μm bifurcating microchannel at a 30° pitch was determined. For different resins with varying Dₚ values ranging from 65 μm to 237 μm, the corresponding minimum Tu for accurate resolution increased with higher Dₚ values. An increase in Dₚ allows UV light to penetrate deeper into the printed part, necessitating a greater amount of resin displacement with each fabricated layer. Precise channel resolution can in certain embodiments include the replacement of resin within a channel before printing each subsequent layer if the original resin had accumulated a dose exceeding a critical threshold, which is denoted as E*. By following a derivation process similar to the Jacobs working curve, the relationship between Tu and threshold dose is expressed as set forth in Equation 2:

$$Turnover Number ( Tu ) = \frac{{D}_{p}}{s} ln ( \frac{{E}_{o}}{{E}^{*}} ) \qquad Equation 2$$

where Eₒ is layer exposure dose and E* is the threshold dose. FIG. 5D shows a plot of the turnover number as a function of resin penetration depth. In accordance with Equation 2, the minimum turnover number required to accurately resolve negative features scales linearly with penetration depth; the slope of this line is

$$\frac{1}{s} ln ( \frac{{E}_{o}}{{E}^{*}} ) .$$

This analysis shows that for a given penetration depth and exposure dose during part fabrication, the minimum resin turnover to achieve high-resolution negative features is determined. Furthermore, from the slope of this line, the value of the critical threshold dose can be extracted for any set of experiment parameters. Specifically, from the slope resin that has accumulated more than 70 percent of the dose should in some instances be cleared to cure a layer.

### High Resolution Polymeric Construction and Applications

Methods for preparing a polymeric structure according to the present disclosure (e.g., iCLIP) can be utilized to fabricate free-form structures having micrometer scale feature resolution in XY and Z coordinates. This process control framework was used to construct various microsystems ranging from personalized medical technologies to microelectromechanical systems (FIG. 6).

Advances in bioengineering and material science have led to the development of personalized medical technologies that enable disease diagnostics and therapeutic delivery at the “point of person”. Among these technologies, microneedles are a promising solution for transdermal drug delivery because they are minimally invasive. Microfluidic elements with microneedle technology is illustrated to provide new fluid management capabilities for transdermal drug delivery and unique fill-finish opportunities of such devices (FIG. 6A and FIG. 6B). An example of microelectromechanical systems fabricated with free-form design geometries is shown in FIG. 6C where a microfluidic inductor is embedded with gallium metal conductive elements. Furthermore, methods described herein can be used to fabricate interlocking vascular perfusion networks for molecular blood transport systems as is shown in FIG. 6E. Systems of the present disclosure have shown the ability to print porous perfusion networks to perform enhanced separations (FIG. 6F).

### CONCLUSIONS

Methods of the present disclosure enables the free-form fabrication of microsystems that uses a fluid control methodology rather than use of optical dyes. This approach can according to certain embodiments resolve microscale negative spaces breaking the relationship between resin penetration depth and negative feature resolution. This overcomes resolution capabilities opening the ability to print high resolution microsystems in materials and designs not before possible. In certain instances, methods include injecting different classes of displacing agents including non-polymerizable fluids such as water and air to allow for resolved negative spaces.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112(f) or 35 U.S.C. § 112(6) is not invoked.

What is claimed is:

1. A method of making a polymeric structure comprising a micro-void space, the method comprising:
a) irradiating a polymerizable composition positioned between a build elevator and a build surface of a liquid interface production module to generate a polymerizable composition comprising a polymerized region of the polymerizable composition comprising a micro-void space in contact with the build elevator and a non-polymerized region of the polymerizable composition in contact with the build surface;
b) displacing the build elevator away from the build surface;
c) contacting the generated micro-void space with a non-reactive composition; and
d) repeating steps a)-c) in a manner sufficient to generate a polymeric structure having a resolved micro-void space.

2. The method according to claim 1, wherein the generated micro-void space comprises one or more microchannels in the polymeric structure which extend through the polymeric structure.

3. The method according to any one of claims 1-2, wherein the method comprises conveying an amount of the polymerizable composition through the generated micro-void space in a manner sufficient to displace polymerized material in the micro-void space.

4. The method according to any one of claims 1-2, wherein the method comprises contacting the generated micro-void space with a non-polymerizable composition.

5. The method according to claim 4, wherein the non-polymerizable composition is continuously contacted with the micro-void space while generating the polymeric structure.

6. The method according to any one of claims 4-5, wherein the non-polymerizable composition is non-reactive with the polymerizable composition of the polymeric structure.

7. The method according to claim 6, wherein the non-polymerizable composition is selected from the group consisting of water, a Newtonian liquid, a shear thinning liquid, a shear thickening liquid, a magnetorheological liquid, an electric field responsive liquid and a gas.

8. The method according to any one of claims 1-7, wherein the method further comprises adding polymerizable composition to the build surface after each displacement of the build elevator away from the build surface.

9. The method according to claim 8, wherein the method further comprises contacting the generated micro-void space with the non-reactive composition after each displacement of the build elevator away from the build surface.

10. A system for making a polymeric structure comprising a micro-void space, the system comprising:
a light source; and
a light interface polymerization module comprising a build elevator and a build surface configured for generating a polymeric structure comprising a resolved micro-void space therein from a polymerizable composition positioned therebetween.

11. The system according to claim 10, wherein the light interface polymerization module is configured for generating a polymeric structure having one or more microchannels in the polymeric structure.

12. The system according to any one of claims 10-11, wherein the system further comprises a processor comprising memory operably coupled to the processor wherein the memory comprises instructions stored thereon, which when executed by the processor, cause the processor to:
a) irradiate a polymerizable composition positioned between a build elevator and a build surface to generate a polymerizable composition comprising a polymerized region of the polymerizable composition comprising a micro-void space in contact with the build elevator and a non-polymerized region of the polymerizable composition in contact with the build surface;
b) displace the build elevator away from the build surface;
c) contact the generated micro-void space with a non-reactive composition; and
d) repeat steps a)-c) in a manner sufficient to generate a polymeric structure having a resolved micro-void space.

13. The system according to claim 12, wherein the memory comprises instructions for conveying an amount of the polymerizable composition through the generated micro-void space in a manner sufficient to displace polymerized material in the micro-void space.

14. The system according to any one of claims 10-12, wherein the memory comprises instructions for contacting the generated micro-void space with a non-polymerizable composition.

15. A polymeric structure comprising a resolved micro-void space comprising a non-polymerizable composition positioned therein.
